SEQUENCE LISTING (28 sequences)

SEQ ID NO: 1 - skipped

SEQ ID NO: 2 - protein, 23 aa, Homo sapiens
AEPRQEFEVMEDHAGTYGLGDRK

SEQ ID NO: 3 - skipped

SEQ ID NO: 4 - protein, 23 aa, Macaca fascicularis
AEPRQEFDVMEDHAGTYGLGDRK

SEQ ID NO: 5-9 - skipped (5 entries)

SEQ ID NO: 10 - protein, 117 aa, Unknown
  Murine species
EVQLVESGGDLAKPGGSLKLSCTASGLIFRSYGMSWVRQTPDKRLEWVATINSGGTYTYYPDSVKGRFTISRDNAKNTLYLQMSSLKSEDTAMYYCANSYSGAMDYWGQGTSVTVSS

SEQ ID NO: 11 - protein, 112 aa, Unknown
  Murine species
DDLLTQTPLSLPVSLGDPASISCRSSQSIVHSNGNTYFEWYLQKPGQSPKLLIYKVSNRFSGVPDRFSGSGSGTDFTLKISRVEAEDLGVYYCFQGSLVPWTFGGGTKLEIK

SEQ ID NO: 12 - protein, 5 aa, Unknown
  Murine species
SYGMS

SEQ ID NO: 13 - protein, 17 aa, Unknown
  Murine species
TINSGGTYTYYPDSVKG

SEQ ID NO: 14 - protein, 8 aa, Unknown
  Murine species
SYSGAMDY

SEQ ID NO: 15 - protein, 16 aa, Unknown
  Murine species
RSSQSIVHSNGNTYFE

SEQ ID NO: 16 - protein, 7 aa, Unknown
  Murine species
KVSNRFS

SEQ ID NO: 17 - protein, 9 aa, Unknown
  Murine species
FQGSLVPWT

SEQ ID NO: 18 - protein, 117 aa, Artificial sequence
  hMTAU heavy chain variable region (VH)
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSS

SEQ ID NO: 19 - protein, 112 aa, Artificial sequence
  hMTAU light chain variable region (VL)
DDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIK

SEQ ID NO: 20 - protein, 5 aa, Artificial sequence
  hMTAU HVR-H1
SYGMS

SEQ ID NO: 21 - protein, 17 aa, Artificial sequence
  hMTAU HVR-H2
TINSGGTYTYYPDSVKG

SEQ ID NO: 22 - protein, 8 aa, Artificial sequence
  hMTAU HVR-H3
SYSGAMDY

SEQ ID NO: 23 - protein, 16 aa, Artificial sequence
  hMTAU HVR-L1
RSSQSIVHSNGNTYLE

SEQ ID NO: 24 - protein, 7 aa, Artificial sequence
  hMTAU HVR-L2
KVSNRFS

SEQ ID NO: 25 - protein, 9 aa, Artificial sequence
  hMTAU HVR-L3
FQGSLVPWT

SEQ ID NO: 26 - protein, 444 aa, Artificial sequence
  hMTAU heavy chain version 1
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 443 aa, Artificial sequence
  hMTAU heavy chain version 2
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 219 aa, Artificial sequence
  hMTAU light chain
DDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0131255 A1
(43) Pub. Date: Apr. 30, 2020

(54) METHODS OF TREATING NEURODEGENERATIVE DISEASES

(71) Applicant: Genentech, Inc., South San Francisco, CA (US)

(72) Inventors: Geoffrey Kerchner, San Francisco, CA (US); Edmond Teng, San Mateo, CA (US)

(73) Assignee: Genentech, Inc., South San Francisco, CA (US)

(21) Appl. No.: 16/576,249

(22) Filed: Sep. 19, 2019

Related U.S. Application Data
(63) Continuation of application No. PCT/US2018/024300, filed on Mar. 26, 2018.
(60) Provisional application No. 62/580,359, filed on Nov. 1, 2017.
(60) Provisional application No. 62/577,559, filed on Oct. 26, 2017.
(60) Provisional application No. 62/532,696, filed on Jul. 14, 2017.
(60) Provisional application No. 62/477,535, filed on Mar. 28, 2017.

(51) Int. Cl. C07K 16/18; A61P 25/28; A61K 9/00; A61K 45/06

(52) U.S. Cl. CPC C07K 16/18; A61P 25/28; A61K 2039/505; A61K 45/06; C07K 2317/24; A61K 9/0019

(57) ABSTRACT

The invention provides methods of treating tauopathies with anti-Tau antibodies.

### CROSS-REFERENCE TO RELATED APPLICATIONS

This application is a continuation of International Application No. PCT/US2018/024300, filed Mar. 26, 2018, which claims the benefit of priority of U.S. Provisional Application No. 62/477,535, filed Mar. 28, 2017; U.S. Provisional Application No. 62/532,696, filed Jul. 14, 2017; U.S. Provisional Application No. 62/577,559, filed Oct. 26, 2017; and U.S. Provisional Application No. 62/580,359, filed Nov. 1, 2017, each of which is incorporated by reference herein in its entirety for any purpose.

### FIELD OF THE INVENTION

The present invention relates to methods of treating neurodegenerative diseases using anti-Tau antibodies.

### BACKGROUND

Neurofibrillary tangles and neuropil threads (NTs) are the major neuropathological hallmarks of Alzheimer's Disease (AD). NTs are composed of the microtubule-associated Tau protein that has undergone post-translational modifications including phosphorylation, and develop by aggregation of hyperphosphorylated Tau conformers. AD shares this pathology with many neurodegenerative tauopathies, in particularly with certain types of frontotemporal dementia (FTD). Tau protein appears to be a major player in the cognitive demise in AD and related neurodegenerative tauopathies.

Therapeutic approaches that target Tau protein are scarce and comprise mainly inhibitors of the kinases that are thought to increase the phosphorylation of Tau to pathological levels, and compounds that block the cytoplasmic aggregation of hyper-phosphorylated Tau protein. These approaches suffer various draw-backs of specificity and efficacy. There is a need for additional therapeutic agents that target the pathological protein conformers that are known or presumed to cause neurodegenerative disorders.

An additional challenge to finding suitable therapeutic agents is the need for the therapeutic agent to gain access to the neurofibrillary tangles and other neuropathological forms of Tau, which are located in the brain. Where the therapeutic agent is a protein, only a very small fraction of peripherally administered therapeutic agent is able to cross the blood brain barrier and reach the presumed location of pathological protein conformers that the agent is intended to target. Therefore, there is a need for treatment regimens capable of delivering therapeutically effective amounts of an agent without causing any deleterious affects.

One particular concern that has arisen in the context of treatments for AD, is the occurrence of imaging abnormalities believed to represent cerebral vasogenic edema and microhemorrhage. These abnormalities have been reported in association with the investigational use of immunotherapy targeting the amyloid beta peptide, possibly by interacting with amyloid beta deposited in or around blood vessels and eliciting an immune response. These abnormalities have been observed to be dose-dependent, arising with more frequency the higher the dose of antibody administered. See, e.g., Sevigny et al., 2016, Nature. 537:50-6. Symptoms, when present in association with such imaging abnormalities, have been reported to include headache, worsening cognitive function, alteration of consciousness, seizures, unsteadiness, and vomiting (Salloway et al. 2009, Neurology 73:2061-70; Sperling et al. 2011, Alzheimers Dement 7:367-85). Tau pathology occurs primarily intracellularly in the cytoplasm of diseased neurons (Braak et al. 2006), and soluble extracellular Tau is found in the cerebrospinal fluid (Blennow and Zetterberg 2009). Unlike AP, Tau is not known to deposit in vascular structures, however, it is not known whether targeting Tau in the human brain could elicit the types of dose-dependent side effects observed with some anti-amyloid beta immunotherapy, especially when administering high doses of Tau immunotherapy.

### SUMMARY

The invention provides methods of treating neurodegenerative diseases with anti-Tau antibodies. As described herein, Applicants have discovered that immunotherapy with antibodies that bind Tau is safe and tolerated even when administered at high doses to both healthy volunteers and patients suffering from AD.

In some embodiments, a method of treating a neurodegenerative disease comprises administering an isolated antibody that binds to human Tau, wherein the antibody binds to monomeric Tau, oligomeric Tau, non-phosphorylated Tau, and phosphorylated Tau. In some embodiments, the antibody binds an epitope within amino acids 2 to 24 of mature human Tau. In some embodiments, the antibody is a monoclonal antibody. In some embodiments, the antibody is a human, humanized, or chimeric antibody. In some embodiments, the antibody is an antibody fragment that binds human Tau. In some embodiments, the human Tau comprises the sequence of SEQ ID NO: 2.

In some embodiments, the antibody comprises HVR-H1 comprising the amino acid sequence of SEQ ID NO: 20; HVR-H2 comprising the amino acid sequence of SEQ ID NO: 21; HVR-H3 comprising the amino acid sequence of SEQ ID NO: 22; HVR-L1 comprising the amino acid sequence of SEQ ID NO: 23; HVR-L2 comprising the amino acid sequence of SEQ ID NO: 24; and HVR-L3 comprising the amino acid sequence of SEQ ID NO: 25.

In some embodiments, the antibody comprises HVR-H1 comprising the amino acid sequence of SEQ ID NO: 20; HVR-H2 comprising the amino acid sequence of SEQ ID NO: 21; HVR-H3 comprising the amino acid sequence of SEQ ID NO: 22; HVR-L1 comprising the amino acid sequence of SEQ ID NO: 23; HVR-L2 comprising the amino acid sequence of SEQ ID NO: 24; and HVR-L3 comprising the amino acid sequence of SEQ ID NO: 25.

In some embodiments, the antibody comprises a heavy chain variable region comprising the amino acid sequence of SEQ ID NO: 18 and a light chain variable region comprising the amino acid sequence of SEQ ID NO: 19.

In some embodiments, the antibody comprises a heavy chain comprising the amino acid sequence of SEQ ID NO: 26 or SEQ ID NO: 27 and a light chain comprising the amino acid sequence of SEQ ID NO: 28. In some embodiments, the hMTAU antibody described herein comprises a heavy chain comprising the amino acid sequence of SEQ ID NO: 27 and a light chain comprising the amino acid sequence of SEQ ID NO: 28.

In some embodiments, an isolated antibody that binds to human Tau is provided, wherein the antibody comprises a heavy chain comprising the amino acid sequence of SEQ ID NO: 26 or SEQ ID NO: 27 and a light chain comprising the amino acid sequence of SEQ ID NO: 28. In some embodiments, an isolated antibody that binds to human Tau is provided, wherein the antibody comprises a heavy chain comprising the amino acid sequence of SEQ ID NO: 26 and a light chain comprising the amino acid sequence of SEQ ID NO: 28. In some embodiments, an isolated antibody that binds to human Tau is provided, wherein the antibody comprises a heavy chain comprising the amino acid sequence of SEQ ID NO: 27 and a light chain comprising the amino acid sequence of SEQ ID NO: 28. In some embodiments, an isolated antibody that binds to human Tau is provided, wherein the antibody comprises a heavy chain consisting of the amino acid sequence of SEQ ID NO: 26 or SEQ ID NO: 27 and a light chain consisting of the amino acid sequence of SEQ ID NO: 28. In some embodiments, an isolated antibody that binds to human Tau is provided, wherein the antibody comprises a heavy chain consisting of the amino acid sequence of SEQ ID NO: 26 and a light chain consisting of the amino acid sequence of SEQ ID NO: 28. In some embodiments, an isolated antibody that binds to human Tau is provided, wherein the antibody comprises a heavy chain consisting of the amino acid sequence of SEQ ID NO: 27 and a light chain consisting of the amino acid sequence of SEQ ID NO: 28. The isolated antibodies described herein may be used in methods of treating a Tau protein associated disease. The isolated antibodies described herein may be used in methods of slowing memory loss or retaining or increasing memory capacity, memory function, or cognitive function in an individual. The isolated antibodies described herein may be used in methods of reducing the level of Tau protein.

In any of the embodiments described herein, the antibody may be an IgG1 or an IgG4 antibody. In any of the embodiments described herein, the antibody may be an IgG4 antibody. In some such embodiments, the antibody comprises M252Y, S254T, and T256E mutations. In any of the embodiments described herein, the antibody may comprise an S228P mutation. In any of the embodiments described herein, the antibody may comprise S228P, M252Y, S254T, and T256E mutations. In any of the embodiments described herein, the antibody may be an IgG4 antibody comprising S228P, M252Y, S254T, and T256E mutations. In some embodiments, the antibody is an antibody fragment. In any of the embodiments described herein, the antibody may be an IgG4 antibody comprising S228P, M252Y, S254T, and T256E mutations, and lacking the C-terminal lysine of the heavy chain constant region. The C-terminal lysine of the heavy chain constant region may be removed, for example, during purification of the antibody or by recombinant engineering of the nucleic acid encoding the antibody such that the C-terminal lysine is not encoded.

In some embodiments, an isolated antibody that binds human Tau is provided, wherein the antibody binds each of monomeric Tau, phosphorylated Tau, non-phosphorylated Tau, and oligomeric Tau with a K_{D} of less than 100 nM, less than 75 nM, or less than 50 nM. In some embodiments, the antibody binds cynomolgus monkey Tau (SEQ ID NO: 4).

In some embodiments, a method of treating a Tau protein associated disease is provided, comprising administering to an individual with a Tau protein associated disease an antibody described herein or a pharmaceutical composition comprising an antibody described herein. In some embodiments, the Tau protein associated disease is a tauopathy. In some embodiments, the tauopathy is a neurodegenerative tauopathy. In some embodiments, the tauopathy is selected from Alzheimer's Disease, amyotrophic lateral sclerosis, Parkinson's disease, Creutzfeldt-Jacob disease, Dementia pugilistica, Down's Syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, the tauopathy is Alzheimer's Disease (AD) or progressive supranuclear palsy. In some embodiments, the AD is early, prodromal, prodromal to mild, mild, mild to moderate or moderate.

In some embodiments, treating a Tau protein associated disease comprises slowing memory loss or retaining or increasing memory capacity, memory function, or cognitive function in the individual.

In some embodiments, a method of slowing memory loss or retaining or increasing memory capacity, memory function, or cognitive function in an individual is provided, comprising administering an antibody described herein or a pharmaceutical composition comprising an antibody described herein.

In some embodiments, memory capacity, memory function, cognitive function, or memory loss is assessed using one or more of Clinical Dementia Rating-Sum of Boxes (CDR-SB), Repeatable Battery for the Assessment of Neuropsychological Status (RBANS), and Alzheimer's Disease Assessment Scale-Cognitive Subscale (ADAS-Cog). In some embodiments, ADAS-Cog is ADAS-Cog 13.

In some embodiments, a decrease in CDR-SB score, or an increase in RBANS score, or a decrease in ADAS-Cog score following administration of one or more doses of the antibody indicates increased cognitive memory capacity in the individual. In some embodiments, a stable CDR-SB score, RBANS score, or ADAS-Cog score following administration of one or more doses of the antibody indicates retained memory capacity, memory function, cognitive function, or slowed memory loss in the individual. In some embodiments, a slowing of the rate of increase of a CDR-SB score or ADAS-Cog score, or a slowing of the rate of decrease of a RBANS score indicates retained memory capacity, memory function, cognitive function, or slowed memory loss in the individual.

In some embodiments, the CDR-SB score, RBANS score, or ADS-Cog score is compared to the respective score at baseline. In some embodiments, baseline is prior to administration of the antibody. In some embodiments, the memory capacity, memory function, cognitive function, or memory loss is assessed at least 13 weeks, at least 24 weeks, at least 25 weeks, at least 37 weeks, at least 49 weeks, at least 61 weeks, at least 69 weeks, at least 73 weeks, at least 85 weeks, at least 97 weeks, at least 109 weeks, at least 121 weeks, at least 133 weeks, at least 145 weeks, at least 157 weeks, or at least 169 weeks after the beginning of treatment with the antibody.

In some embodiments, a method of reducing the level of Tau protein, non-phosphorylated Tau protein, phosphorylated Tau protein, or hyperphosphorylated Tau protein in an individual is provided, comprising administering an antibody described herein or a pharmaceutical composition comprising an antibody described herein.

In some embodiments, an isolated antibody described herein is provided for use as a medicament. In some embodiments, an isolated antibody described herein is provided for use in treating a tauopathy in an individual. In some embodiments, the tauopathy is a neurodegenerative tauopathy. In some embodiments, the tauopathy is selected from Alzheimer's Disease, amyotrophic lateral sclerosis, Parkinson's disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, the tauopathy is Alzheimer's Disease (AD) or progressive supranuclear palsy. In some embodiments, the AD is AD is early, prodromal, prodromal to mild, mild, mild to moderate or moderate.

In some embodiments, an isolated antibody described herein is provided for use in slowing memory loss or retaining or increasing memory capacity, memory function, or cognitive function in an individual. In some embodiments, an isolated antibody described herein is provided for use in reducing the level of Tau protein, phosphorylated Tau protein, non-phosphorylated Tau protein, or hyperphosphorylated Tau protein in an individual.

In some embodiments, use of an antibody described herein is provided for manufacture of a medicament for treating a Tau protein associated disease in an individual. In some embodiments, the Tau protein associate disease is a tauopathy. In some embodiments, the tauopathy is a neurodegenerative tauopathy. In some embodiments, the tauopathy is selected from Alzheimer's Disease, amyotrophic lateral sclerosis, Parkinson's disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, the tauopathy is Alzheimer's disease or progressive supranuclear palsy. In some embodiments, the AD is AD is early, prodromal, prodromal to mild, mild, mild to moderate or moderate.

In some embodiments, use of an antibody described herein is provided for manufacture of a medicament for slowing memory loss or retaining or increasing memory capacity, memory function, or cognitive function in an individual.

In some embodiments, a method of detecting neurofibrillary tangles, neutrophil threads, or dystrophic neuritis is provided, comprising contacting a sample with an antibody described herein. In some embodiments, the sample is a brain sample, a cerebrospinal fluid sample, or a blood sample.

In any of the embodiments described herein, a method or use may comprise administering an antibody described herein in combination with at least one additional therapy. Non-limiting examples of additional therapies include neurological drugs, corticosteroids, antibiotics, antiviral agents, and other therapeutic agents. Such other therapeutic agents include, but are not limited to, other anti-Tau antibodies, antibodies against amyloid-beta, antibodies against a beta-secretase (“BACE”) such as antibodies against beta-secretase 1 (“BACE1”) or antibodies against beta-secretase 2 (“BACE2”), and inhibitors of a beta-secretase, such as inhibitors of beta-secretase 1 or inhibitors of beta-secretase 2.

In some embodiments, the antibody is administered at a dose of 225 mg, 675 mg, 1200 mg, 1500 mg, 2100 mg, 4200 mg, 4500 mg, 8100 mg, 8400 mg, or 16800 mg. In some embodiments, the antibody is administered at a dose between 225 mg and 1000 mg. In some embodiments, the antibody is administered at a dose between 225 mg and 600 mg. In some embodiments, the antibody is administered at a dose between 600 mg and 1000 mg. In some embodiments, the antibody is administered at a dose between 1000 mg and 2000 mg. In some embodiments, the antibody is administered at a dose between 2000 mg and 3000 mg. In some embodiments, the antibody is administered at a dose between 3000 mg and 4000 mg. In some embodiments, the antibody is administered at a dose between 4000 mg and 16800 mg. In some instances, the antibody may be administered at a dose between 4000 mg and 8500 mg. In some embodiments, the antibody is administered at a dose between 4000 mg and 4500 mg, 4000 mg and 5000 mg, 4500 mg and 5000 mg, 5000 mg and 5500 mg, 5500 mg and 6000 mg, 6000 mg and 6500 mg, 6500 mg and 7000 mg, 7000 mg and 7500 mg, 7500 mg and 8000 mg, or 8000 mg and 8500 mg.

In some embodiments, the antibody is administered at a dose between 2.5 mg/kg and 5 mg/kg, 5 mg/kg and 10 mg/kg, 10 mg/kg and 15 mg/kg, 15 mg/kg and 20 mg/kg, 20 mg/kg and 30 mg/kg, 30 mg/kg and 40 mg/kg, 40 mg/kg and 50 mg/kg, 50 mg/kg and 60 mg/kg, 50 mg/kg and 240 mg/kg. In some instances, the antibody is administered at a dose between 60 mg/kg and 120 mg/kg. In some embodiments, the antibody is administered at a dose between 60 mg/kg and 70 mg/kg, 70 mg/kg and 80 mg/kg, 80 mg/kg and 90 mg/kg, 90 mg/kg and 100 mg/kg, 100 mg/kg and 110 mg/kg, or 110 mg/kg and 120 mg/kg.

In some embodiments, the methods and uses described herein comprise administering the antibody once every 2, 4, 5, 6, 7, or 8 weeks. In some instances, the antibody is administered twice every 2, 4, 5, 6, 7, or 8 weeks.

In some embodiments, the methods and uses described herein comprise administering the antibody subcutaneously. In some instances, the methods and uses comprise administering the antibody intravenously.

### BRIEF DESCRIPTION OF THE FIGURES

FIG. 1 shows the overall study design, including the single dose escalation stage and the multiple dose stage of the clinical study in Example 1. Abbreviations used in the figure as follows: DLAE, dose-limiting adverse event; HV, healthy volunteers; AD, Alzheimer's Disease; IV, intravenous; SC, subcutaneous; CSF, cerebrospinal fluid.

FIG. 2 provides mean (±SD) concentration-time profiles of hMTAU in serum after single dose IV or SC administration.

FIG. 3 provides mean (±SD) concentration-time profiles of hMTAU in CSF after single dose 2100 and 8400 mg IV administration.

FIG. 4 provides mean (±SD) concentration-time profiles of hMTAU in serum after single or multiple dose IV or SC administration.

FIG. 5 provides mean (±SD) concentration-time profiles of hMTAU in CSF after single dose 2100 and 8400 mg IV administration and multiple dose 8400 mg IV administration.

FIG. 6 provides mean (±SD) concentration-time profiles of hMTAU in serum (left axis) and mean (±SD) concentration-time profiles of TAU in plasma (right axis) after four weekly doses of hMTAU at 8400 mg IV. PK (pharmacokinetics) and PD (pharmacodymanics) concentration-time profiles are shown. AD=patients with Alzheimer's disease; HV=healthy volunteers.

FIG. 7 shows a design for a clinical study of hMTAU in prodromal-to-mild Alzheimer's disease patients.

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-Tau antibody” and “an antibody that binds to Tau” refer to an antibody that is capable of binding Tau with sufficient affinity such that the antibody is useful as a diagnostic and/or therapeutic agent in targeting Tau. In some embodiments, the extent of binding of an anti-Tau antibody to an unrelated, non-Tau protein is less than about 10% of the binding of the antibody to Tau as measured, e.g., by a radioimmunoassay (MA). In certain embodiments, an antibody that binds to Tau has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³M, e.g., from 10⁻⁹M to 10⁻¹³M). In certain embodiments, an anti-Tau antibody binds to an epitope of Tau that is conserved among Tau from different species. The term “anti-Tau antibody” and “antibody that binds to Tau,” as used herein, refers to an antibody that binds monomeric Tau, oligomeric Tau, and/or phosphorylated Tau, unless specifically indicated otherwise. In some such embodiments, the anti-Tau antibody binds to monomeric Tau, oligomeric Tau, non-phosphorylated Tau, and phosphorylated Tau with comparable affinities, such as with affinities that differ by no more than 50-fold from one another. In some embodiments, an antibody that binds monomeric Tau, oligomeric Tau, non-phosphorylated Tau, and phosphorylated Tau is referred to as a “pan-Tau antibody.”

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Tau antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “Tau,” as used herein, refers to any native Tau protein from any vertebrate source, including mammals such as primates (e.g., humans) and rodents (e.g., mice and rats), unless otherwise indicated. The term encompasses “full-length,” unprocessed Tau as well as any form of Tau that results from processing in the cell. The term also encompasses naturally occurring variants of Tau, e.g., splice variants or allelic variants.

The term “pTau,” as used herein, refers to Tau in which a serine, a threonine or a tyrosine residue is phosphorylated by a protein kinase by the addition of a covalently bound phosphate group. In some embodiments, pTau is phosphorylated on a serine or on a threonine residue. In some embodiments, pTau is phosphorylated on Serine at position 409 and/or Serine at position 404. In some embodiments, pTau is phosphorylated on Serine at position 409.

The terms “soluble Tau” or “soluble Tau protein,” as used herein, refer to proteins consisting of both completely solubilized Tau protein/peptide monomers or of Tau-like peptides/proteins, or of modified or truncated Tau peptides/proteins or of other derivates of Tau peptides/proteins monomers, and of Tau protein oligomers. “Soluble Tau” excludes particularly neurofibrillary tangles (NFT).

The term “insoluble Tau,” as used herein, refers to multiple aggregated monomers of Tau peptides or proteins, or of Tau-like peptides/proteins, or of modified or truncated Tau peptides/proteins or of other derivates of Tau peptides/proteins forming oligomeric or polymeric structures which are insoluble both in vitro in aqueous medium and in vivo in the mammalian or human body more particularly in the brain, but particularly to multiple aggregated monomers of Tau or of modified or truncated Tau peptides/proteins or of derivatives thereof, which are insoluble in the mammalian or human body more particularly in the brain, respectively. “Insoluble Tau” particularly includes neurofibrillary tangles (NFT).

The terms “monomeric Tau” or “Tau monomer,” as used herein, refer to completely solubilized Tau proteins without aggregated complexes in aqueous medium.

The terms “aggregated Tau”, “oligomeric Tau” and “Tau oligomer,” as used herein, refer to multiple aggregated monomers of Tau peptides or proteins, or of Tau-like peptides/proteins, or of modified or truncated Tau peptides/proteins or of other derivates of Tau peptides/proteins forming oligomeric or polymeric structures which are insoluble or soluble both in vitro in aqueous medium and in vivo in the mammalian or human body more particularly in the brain, but particularly to multiple aggregated monomers of Tau or of modified or truncated Tau peptides/proteins or of derivatives thereof, which are insoluble or soluble in the mammalian or human body more particularly in the brain, respectively.

The terms “pTau PHF”, “PHF”, and “paired helical filaments,” are used herein synonymously, refer to pairs of filaments wound into helices with a periodicity of 160 nm visible on electron microscopy. Width varies between 10 and 22 nm. PHF are the predominant structures in neurofibrillary tangles of Alzheimer's Disease (AD) and neuropil threads. PHF may also be seen in some but not all dystrophic neurites associated with neuritic plaques. The major component of PHF is a hyperphosphorylated form of microtubule-associated protein tau. PHF may be partially composed of disulfide-linked antiparallel hyper-phosphorylated Tau proteins. PHF Tau may be truncated of its C-terminal 20 amino acid residues. The mechanisms underlying PHF formation are uncertain but hyper-phosphorylation of Tau may disengage it from microtubules, increasing the soluble pool of Tau from which PHF can be formed inside neurons.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

A “fixed” or “flat” dose of a therapeutic agent refers to a dose that is administered to a human patient without regard for the weight (WT) or body surface area (BSA) of the patient. The fixed or flat dose is therefore not provided as a mg/kg dose or a mg/m2 dose, but rather as an absolute amount of the therapeutic agent.

The term “early Alzheimer's Disease” or “early AD” as used herein (e.g., a “patient diagnosed with early AD” or a “patient suffering from early AD”) includes patients with mild cognitive impairment, such as a memory deficit, due to AD and patients having AD biomarkers, for example amyloid positive patients, patients having a positive florbetapir PET scan, or patients having a positive Tau PET scan.

The term “MMSE” refers to the Mini Mental State Examination, which provides a score between 1 and 30. See Folstein, et al., 1975, J. Psychiatr. Res. 12:189-98. Scores of 26 and lower are generally considered to be indicative of a deficit. The lower the numerical score on the MMSE, the greater the tested patient's deficit or impairment relative to another individual with a lower score. An increase in MMSE score may be indicative of improvement in the patient's condition, whereas a decrease in MMSE score may denote worsening in the patient's condition.

The term “CDR-SB” refers to the Clinical Dementia Rating-Sum of Boxes, which provides a score between 0 and 18. See, e.g. O'Bryant et al., 2008, Arch Neurol 65: 1091-1095. CDR-SB score is based on semi-structured interviews of patients and caregiver informants, and yields five degrees of impairment in performance for each of six categories of cognitively-based functioning: memory, orientation, judgment and problem solving, community affairs, home and hobbies, and personal care. Each category is scored from 0-3 (the five degrees are 0, 0.5, 1, 2, and 3). The sum of the score for the six categories is the CDR-SB score. A decrease in CDR-SB score may be indicative of improvement in the patient's condition, whereas an increase in CDR-SB score may be indicative of worsening of the patient's condition. In some embodiments, a stable CDR-SB score may be indicative of a slowing, delay, or cessation of the progression of AD, or a lack of appearance of new clinical, functional, or cognitive symptoms or impairments, or an overall stabilization of disease activity.

The term “RBANS” refers to Repeatable Battery for the Assessment of Neuropsychological Status, which consists of twelve subtests that are combined to provide five indices, one for each of the five domains tested (immediate memory, visuospatial/constructional, language, attention, and delayed memory). See, e.g., Randolph et al., 1998, J Clin Exp Neuropsychol 20: 310-319. Extensive normative values are provided in the testing manuals. An increase in RBANS score may be indicative of improvement in the patient's condition, whereas a decrease in RBANS score may be indicative of worsening of the patient's condition. In some embodiments, a stable RBANS score may be indicative of a slowing, delay, or cessation of the progression of AD, or a reduction in clinical or cognitive decline.

The term “ADAS-Cog 13” refers to the version of the Alzheimer's Disease Assessment Scale-Cognitive Subscale that includes 13 items. See, e.g., Rosen et al., 1984, Amer. J. Psych. 141: 1356-1364; Mohs et al., 1997, Alzheimer's Disease Assoc. Disorders, 11(2): S13-S21. The ADAS-Cog 13 is a multi-part cognitive assessment that assesses multiple cognitive domains, including memory, naming, word finding, comprehension, praxis, attention, orientation, and spontaneous speech. The ADAS-Cog 13 is based on the ADAS-Cog and additionally includes delayed word recall and a number cancellation task. The ADAS-Cog 13 produces scores up to 85 points. A decrease in ADAS-Cog 13 score may be indicative of improvement in the patient's condition, whereas an increase in ADAS-Cog 13 score may be indicative of worsening of the patient's condition. In some embodiments, a stable ADAS-Cog 13 score may be indicative of a slowing, delay, or cessation of the progression of AD, or a reduction in clinical or cognitive decline.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

Methods of treating tauopathies with antibodies that bind Tau are provided. In particular, methods of treating tauopathies with antibodies that bind Tau have been found to be safe and tolerated even when administered at high doses. As such, methods of treating tauopathies with antibodies that bind Tau at doses ranging from 225 mg to 16800 mg are provided. In some embodiments, an antibody binds monomeric Tau, oligomeric Tau, non-phosphorylated Tau, and phosphorylated Tau. In some embodiments, the antibody binds to an epitope within amino acids 2 to 24 of mature human Tau. In some embodiments, the antibody binds to an epitope within Tau amino acids 2 to 24 and binds monomeric Tau, oligomeric Tau, non-phosphorylated Tau, and phosphorylated Tau. In some embodiments, the antibody binds an epitope of human Tau having, or consisting of, the sequence AEPRQEFEVMEDHAGTYGLGDRK (SEQ ID NO: 2). In some embodiments, the antibody binds an epitope of cynomolgus monkey Tau having, or consisting of, the sequence AEPRQEFDVMEDHAGTYGLGDRK (SEQ ID NO: 4).

A. Exemplary Anti-Tau Antibodies

In some embodiments, an anti-Tau antibody comprises (a) HVR-H1 comprising the amino acid sequence of SEQ ID NO: 20; (b) HVR-H2 comprising the amino acid sequence of SEQ ID NO: 21; and (c) HVR-H3 comprising the amino acid sequence of SEQ ID NO: 22. In some embodiments, an anti-Tau antibody comprises at least one, two, or three HVRs selected from (a) HVR-H1 comprising the amino acid sequence of SEQ ID NO: 20; (b) HVR-H2 comprising the amino acid sequence of SEQ ID NO: 21; and (c) HVR-H3 comprising the amino acid sequence of SEQ ID NO: 22. In some embodiments, an anti-Tau antibody comprises (a) HVR-H1 comprising the amino acid sequence of SEQ ID NO: 20; (b) HVR-H2 comprising the amino acid sequence of SEQ ID NO: 21; (c) HVR-H3 comprising the amino acid sequence of SEQ ID NO: 22; (d) HVR-L1 comprising the amino acid sequence of SEQ ID NO: 23; (e) HVR-L2 comprising the amino acid sequence of SEQ ID NO: 24; and (f) HVR-L3 comprising the amino acid sequence of SEQ ID NO: 25.

In some embodiments, an anti-Tau antibody comprises (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14. In some embodiments, an anti-Tau antibody comprises at least one, two, or three HVRs selected from (a) HVR-H1 comprising the amino acid sequence of SEQ ID NO: 12; (b) HVR-H2 comprising the amino acid sequence of SEQ ID NO: 13; and (c) HVR-H3 comprising the amino acid sequence of SEQ ID NO: 14. In some embodiments, an anti-Tau antibody comprises (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising the amino acid sequence of SEQ ID NO: 17.

In some embodiments, an anti-Tau antibody is humanized. In some embodiments, an anti-Tau antibody comprises HVRs as in an above embodiment, and further comprises an acceptor human framework, e.g. a human immunoglobulin framework or a human consensus framework.

In some embodiments, the antibody comprises the VH and VL sequences in SEQ ID NO: 18 and SEQ ID NO: 19, respectively, including post-translational modifications of those sequences, if any.

In some embodiments, the antibody comprises the VH and VL sequences in SEQ ID NO: 10 and SEQ ID NO: 11, respectively, including post-translational modifications of those sequences, if any.

In some embodiments, an anti-Tau antibody is provided, wherein the antibody comprises a heavy chain comprising the amino acid sequence of SEQ ID NO: 26 or SEQ ID NO: 27 and a light chain comprising the amino acid sequence of SEQ ID NO: 28. In some embodiments, an anti-Tau antibody is provided, wherein the antibody comprises a heavy chain consisting of the amino acid sequence of SEQ ID NO: 26 or SEQ ID NO: 27 and a light chain consisting of the amino acid sequence of SEQ ID NO: 28. In some embodiments, an anti-Tau antibody is provided, wherein the antibody comprises a heavy chain comprising the amino acid sequence of SEQ ID NO: 27 and a light chain comprising the amino acid sequence of SEQ ID NO: 28. In some embodiments, an anti-Tau antibody is provided, wherein the antibody consists of a heavy chain comprising the amino acid sequence of SEQ ID NO: 27 and a light chain consisting of the amino acid sequence of SEQ ID NO: 28. In some embodiments, an anti-Tau antibody is provided, wherein the antibody consists of a heavy chain comprising the amino acid sequence of SEQ ID NO: 26 and a light chain consisting of the amino acid sequence of SEQ ID NO: 27. In a further aspect of the invention, an anti-Tau antibody according to any of the above embodiments is a monoclonal antibody, including a chimeric, humanized or human antibody. In one embodiment, an anti-Tau antibody is an antibody fragment, e.g., a Fv, Fab, Fab′, scFv, diabody, or F(ab′)₂ fragment. In another embodiment, the antibody is a full length antibody, e.g., an intact IgG1 or IgG4 antibody or other antibody class or isotype as defined herein.

In a further aspect, an anti-Tau antibody according to any of the above embodiments may incorporate any of the features, singly or in combination, as described in Sections 1-5 below:

1. Antibody Affinity

In certain embodiments, an antibody provided herein has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

In some embodiments, K_{D} is measured by a radiolabeled antigen binding assay (MA). In some embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE e-3000 (BIAcore, Inc., Piscataway, N.J.) is performed at 25° C. with immobilized antigen CMS chips at ˜10 resonance units (RU). In some embodiments, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 μg/ml (˜0.2 μM) before injection at a flow rate of 5 μl/minute to achieve approximately 10 resonance units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for Tau and the other is for any other antigen. In certain embodiments, one of the binding specificities is for Tau and the other is for amyloid beta. In certain embodiments, bispecific antibodies may bind to two different epitopes of Tau. Bispecific antibodies may also be used to localize cytotoxic agents to cells which express Tau. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to Tau as well as another, different antigen (see, US 2008/0069820, for example).

5. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| | Exemplary | Preferred |
|---|---|---|
| Original Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (e.g., U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In some embodiments, isolated nucleic acid encoding an anti-Tau antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g. a Chinese Hamster Ovary (CHO) cell or lymphoid cell (e.g., Y0, NS0, Sp20 cell). In some embodiments, a method of making an anti-Tau antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Tau antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

C. Assays

Anti-Tau antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with an antibody described herein for binding to Tau. In certain embodiments, such a competing antibody binds to the same epitope (e.g., a linear or a conformational epitope) that is bound by MTAU or hMTAU. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Tau (such as monomeric Tau) is incubated in a solution comprising a first labeled antibody that binds to Tau (e.g., any antibody described herein, such as MTAU or hMTAU) and a second unlabeled antibody that is being tested for its ability to compete with the first antibody for binding to Tau. The second antibody may be present in a hybridoma supernatant. As a control, immobilized Tau is incubated in a solution comprising the first labeled antibody but not the second unlabeled antibody. After incubation under conditions permissive for binding of the first antibody to Tau, excess unbound antibody is removed, and the amount of label associated with immobilized Tau is measured. If the amount of label associated with immobilized Tau is substantially reduced in the test sample relative to the control sample, then that indicates that the second antibody is competing with the first antibody for binding to Tau. See Harlow and Lane (1988) Antibodies: A Laboratory Manual ch. 14 (Cold Spring Harbor Laboratory, Cold Spring Harbor, N.Y.).

2. Activity Assays

In one aspect, assays are provided for identifying anti-Tau (e.g., pan-Tau) antibodies thereof having biological activity. Biological activity may include, e.g., binding of such antibodies to multiple forms of Tau (e.g., monomeric Tau, oligomeric Tau, non-phosphorylated Tau, and phosphorylated Tau) and reducing the level of Tau protein (e.g., total Tau, total soluble Tau, soluble non-phosphorylated Tau, soluble phosphorylated Tau, total insoluble Tau, insoluble non-phosphorylated Tau, insoluble phosphorylated Tau, hyperphosphorylated Tau, or paired helical filaments containing hyperphosphorylated Tau, in the brain, e.g., in the brain cortex and/or hippocampus). Antibodies having such biological activity in vivo and/or in vitro are also provided.

In certain embodiments, an antibody of the invention is tested for such biological activity. For example, an animal model of tauopathy, such as a Tau transgenic mice (e.g., P301L), can be used to detect binding of anti-Tau antibodies to brain sections, and for example, to neurofibrillary tangles in the brains of the transgenic mice. Further, an animal model of tauopathy, such as a Tau transgenic mice (e.g., P301L), can be treated with anti-Tau antibodies and experimental techniques known in the art can be used to assess whether such treatment reduces the level of Tau protein (e.g., total Tau, total soluble Tau, soluble phosphorylated Tau, soluble non-phosphorylated Tau, total insoluble Tau, insoluble phosphorylated Tau, insoluble non-phosphorylated Tau, hyperphosphorylated Tau, or paired helical filaments containing hyperphosphorylated Tau) in the mouse brain (e.g., in the brain cortex and/or hippocampus).

D. Immunoconjugates

The invention also provides immunoconjugates comprising an anti-Tau antibody herein conjugated to one or more other therapeutic agents or radioactive isotopes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinyl sulfone)benzoate) which are commercially available (e.g., from Pierce Biotechnology, Inc., Rockford, Ill., U.S.A).

E. Pharmaceutical Formulations

Pharmaceutical formulations of an anti-Tau antibody as described herein are prepared by mixing such antibody having the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

F. Therapeutic Methods and Compositions

Any of the anti-Tau antibodies provided herein may be used in therapeutic methods.

In one aspect, an anti-Tau antibody for use as a medicament is provided. In further aspects, an anti-Tau antibody for use in treating a Tau protein associated disease or disorder is provided. In some embodiments, an anti-Tau antibody for use in treating diseases or disorders caused by or associated with the formation of neurofibrillary tangles or neuropil threads is provided. In particular embodiments, an anti-Tau antibody for use in treating a tauopathy such as a neurodegenerative tauopathy is provided. Exemplary Tau protein associated diseases or disorders that can be treated with anti-Tau antibodies include, without limitation, Alzheimer's Disease, amyotrophic lateral sclerosis, Parkinson's disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, an anti-Tau antibody for use in treating Alzheimer's Disease (AD) is provided herein. In some embodiments, an anti-Tau antibody for use in treating moderate AD, mild to moderate AD, mild AD, early AD, prodromal to mild AD, or prodromal AD is provided herein. Further, Tau protein associated diseases or disorders that can be treated with an anti-Tau antibody include diseases or disorders that are manifested in an impairment or loss of a cognitive function such as reasoning, situational judgement, memory capacity, learning, and/or special navigation. In certain embodiments, an anti-Tau antibody for use in a method of treatment is provided. In certain embodiments, the invention provides an anti-Tau antibody for use in a method of treating an individual, having any one of the Tau associated diseases or disorders described above, comprising administering to the individual an effective amount of the anti-Tau antibody. In one such embodiment, the method further comprises administering to the individual an effective amount of at least one additional therapeutic agent, e.g., as described below.

In some embodiments, an antibody of the invention is used to treat an individual having an MMSE score of between 16 and 28, between 16 and 18, between 16 and 20, between 20 and 30, between 20 and 26, between 24 and 30, between 21 and 26, between 22 and 26, between 22 and 28, between 23 and 26, between 24 and 26, or between 25 and 26. In some embodiments, the patient has an MMSE score between 22 and 26. As used herein, an MMSE score between two numbers includes the numbers at each end of the range. For example, an MMSE score between 22 and 26 includes MMSE scores of 22 and 26.

In some embodiments, an antibody of the invention is used to treat an individual who is ‘tau positive,’ e.g., a patient having brain tau deposits that are typical of Tau protein associated disorders, e.g., a patient having a positive Tau positron emission tomography (PET) scan.

In further embodiments, the invention provides an anti-Tau antibody for use in reducing the levels of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in an individual. In some embodiments, the invention provides an anti-Tau antibody for use in reducing the levels of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in an individual as measured by Tau PET scan. For example, such reduction can occur in the brain (e.g., in the brain cortex and/or hippocampus). In some embodiments, the invention provides an anti-Tau antibody for use in reducing the levels of phosphorylated Tau, including soluble phosphorylated Tau. In some embodiments, the invention provides an anti-Tau antibody for use in reducing the levels of aggregated Tau. In some embodiments, the invention provides an anti-Tau antibody for use in reducing the levels of insoluble Tau (e.g., insoluble phosphorylated Tau). In some embodiments, the invention provides an anti-Tau antibody for use in reducing the levels of hyperphosphorylated Tau. In some embodiments, the invention provides an anti-Tau antibody for use in reducing the levels of paired helical filaments (e.g., paired helical filaments containing hyperphosphorylated Tau) in a brain tissue (e.g., in the brain cortex and/or hippocampus). In certain embodiments, the invention provides an anti-Tau antibody for use in a method of reducing the levels of Tau protein (e.g., total Tau, total soluble Tau, soluble phosphorylated Tau, total insoluble Tau, insoluble phosphorylated Tau, hyperphosphorylated Tau, or paired helical filaments containing hyperphosphorylated Tau) in the brain (e.g., in the brain cortex and/or hippocampus) in an individual comprising administering to the individual an effective amount of the anti-Tau antibody to reduce the levels of Tau protein. An “individual” according to any of the above embodiments is a mammal, preferably a human. In some embodiments, a reduction in the level of Tau protein is determined by measuring the density and/or extent of Tau pathology and/or aggregated Tau. As such, reduced density or extent of Tau pathology and/or aggregated Tau (measured, e.g., by positron emission tomography imaging) is considered indicative of a reduction in the level of Tau. The level of Tau protein, non-phosphorylated Tau protein, phosphorylated Tau protein, or hyperphosphorylated Tau protein can be measured by positron emission tomography (PET) or by analysis of cerebrospinal fluid, such as cerebrospinal fluid obtained via lumbar puncture. In some embodiments, a reduction in the level of Tau protein is determined by measuring the level of a Tau fragment.

In some embodiments, the invention provides an anti-Tau antibody for use in modulating the levels of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau), for example, in the brain (e.g., in the brain cortex and/or hippocampus) of an individual.

In a further aspect, the invention provides for the use of an anti-Tau antibody in the manufacture or preparation of a medicament. In some embodiments, the medicament is for treatment of a Tau protein associated disease or disorder. The Tau protein associated disease or disorder can be a disease or disorder caused by or associated with the formation of neurofibrillary tangles or neuropil threads. In some embodiments, the medicament is for treatment of a tauopathy such as a neurodegenerative tauopathy. In specific embodiments, the medicament is for treatment of diseases or disorders selected from the group consisting of: Alzheimer's Disease (AD), Creutzfeldt-Jacob disease, Dementia pugilistica, Down's Syndrome, Gerstmann-Str-ussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, the medicament is for treatment of AD. In some embodiments, the medicament is for treatment of a Tau associated disease or disorder that is manifested in an impairment or loss of a cognitive function such as reasoning, situational judgement, memory capacity, learning, or special navigation. In a further embodiment, the medicament is for use in a method of treating one of the above-listed diseases (e.g., a tauopathy such as AD) comprising administering to an individual having such disease an effective amount of the medicament. In one such embodiment, the method further comprises administering to the individual an effective amount of at least one additional therapeutic agent, e.g., as described below.

In a further embodiment, the medicament is for reducing the levels of Tau protein (e.g., total Tau, total soluble Tau, soluble non-phorphorylated Tau, soluble phosphorylated Tau, total insoluble Tau, aggregated Tau, insoluble phosphorylated Tau, insoluble non-phorphorylated Tau, hyperphosphorylated Tau, or paired helical filaments containing hyperphosphorylated Tau). For example, such reducing of Tau protein can be observed in the brain (e.g., in the brain cortex and/or hippocampus) or in cerebrospinal fluid of an individual. In some embodiments, the medicament is for reducing the levels of paired helical filaments. In a further embodiment, the medicament is for use in a method of reducing the levels of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in an individual comprising administering to the individual an effective amount of the medicament to reducing the levels of Tau protein. An “individual” according to any of the above embodiments is a mammal, preferably, a human.

In a further aspect, the invention provides a method for treating a Tau protein associated disease or disorder. Tau protein associated disease or disorder that can be treated in accordance with the methods provided herein include diseases or disorders caused by or associated with the formation of neurofibrillary tangles or neuropil threads. In particular embodiments, the invention provides a method for treating a tauopathy such as a neurodegenerative tauopathy. In specific embodiments, the invention provides a method for treating a disease or disorder selected from the group consisting of: Alzheimer's Disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, the invention provides a method for treating Alzheimer's Disease (AD). In particular embodiments, the invention provides a method for treating a Tau protein associated disease or disorder that is manifested in an impairment or loss of a cognitive function such as reasoning, situational judgement, memory capacity, learning, or special navigation. In some embodiments, the method comprises administering to an individual, having any one of the diseases or disorders described above, an effective amount of an anti-Tau antibody. In one such embodiment, the method further comprises administering to the individual an effective amount of at least one additional therapeutic agent, e.g., as described below. In some embodiments, the method comprises administering to an individual having one of the diseases described herein an effective amount of an anti-Tau antibody. In one such embodiment, the method further comprises administering to the individual an effective amount of at least one additional therapeutic agent, as described below. An “individual” according to any of the above embodiments may be a human.

In a further aspect, the invention provides a method for reducing the levels of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in an individual. For example, such reducing of the levels of Tau protein can be observed in the brain (e.g., brain cortex and/or hippocampus) or cerebrospinal fluid of an individual. In some embodiments, the invention provides a method for reducing the levels of paired helical filaments. In some embodiments, the method comprises administering to the individual an effective amount of an anti-Tau antibody to reduce the levels of Tau protein. An “individual” according to any of the above embodiments may be a human.

In some aspects, the invention provides a method for alleviating one or more symptoms of a Tau protein associated disease or disorder; or an anti-Tau antibody or a medicament comprising anti-Tau antibody for alleviating one or more symptoms of a Tau protein associated disease or disorder (such as any of the diseases or disorders described herein, for example, AD). In some aspects, the invention provides a method for reducing the number of symptoms or the severity of one or more symptoms of a Tau protein associated disease or disorder; or an anti-Tau antibody or a medicament comprising anti-Tau antibody for reducing the number of symptoms or the severity of one or more symptoms of a Tau protein associated disease or disorder (such as any of the diseases or disorders described herein, for example, AD). In a particular embodiment, the symptom of a Tau protein associated disease or disorder is an impairment in cognition. In a specific embodiment, the symptom of a Tau protein associated disease or disorder is an impairment in learning and/or memory. In a specific embodiment, the symptom of a Tau protein associated disease or disorder is a long-term memory loss. In a specific embodiment, the symptom of a Tau protein associated disease or disorder is dementia. In some embodiments, the symptom of a Tau protein associated disease or disorder is confusion, irritability, aggression, mood swings, or a language impairment. In some embodiments, the symptom of a Tau protein associated disease or disorder is an impairment or loss of one or more cognitive functions such as reasoning, situational judgment, memory capacity, and/or learning. The methods provided herein comprise administration of an amount (e.g., therapeutically effective amount) of an anti-Tau antibody to an individual (e.g., who displays one or more symptoms of a Tau protein associated disease or disorder).

In specific aspects, the invention provides a method for retaining or increasing memory capacity, memory function, or cognitive function, or for slowing down memory loss associated with a Tau protein associated disease or disorder; or an anti-Tau antibody or a medicament comprising anti-Tau antibody for retaining or increasing memory capacity, memory function, or cognitive function, or for slowing down memory loss associated with a Tau protein associated disease or disorder (such as any of the diseases or disorders described herein, for example, AD). The methods provided herein comprise administration of an amount (e.g., therapeutically effective amount) of an anti-Tau antibody to an individual (e.g., who displays one or more symptoms of memory loss or a decrease of memory capacity).

In some aspects, the invention provides a method for decreasing the rate of progression of a Tau protein associated disease or disorder; or an anti-Tau antibody or a medicament comprising anti-Tau antibody for decreasing the rate of progression of a Tau protein associated disease or disorder (such as any of the diseases or disorders described herein, for example, AD). The methods provided herein comprise administration of an amount (e.g., therapeutically effective amount) of an anti-Tau antibody to an individual (e.g., who displays one or more symptoms of a Tau protein associated disease or disorder).

In some aspects, the invention provides a method for preventing the development of a Tau protein associated disease or disorder; or an anti-Tau antibody or a medicament comprising anti-Tau antibody for preventing the development of a Tau protein associated disease or disorder (such as any of the diseases or disorders described herein, for example, AD). The methods provided herein comprise administration of an amount (e.g., therapeutically effective amount) of an anti-Tau antibody to an individual (e.g., who is at risk of a Tau protein associated disease or disorder).

In some aspects, the invention provides a method for delaying the development of a Tau protein associated disease or disorder; or an anti-Tau antibody or a medicament comprising anti-Tau antibody for delaying the development of a Tau protein associated disease or disorder (such as any of the diseases or disorders described herein, for example, AD). The methods provided herein comprise administration of an amount (e.g., therapeutically effective amount) of an anti-Tau antibody to an individual (e.g., who displays one or more symptoms of a Tau protein associated disease or disorder).

In a further aspect, the invention provides pharmaceutical formulations comprising any of the anti-Tau antibodies provided herein, e.g., for use in any of the above therapeutic methods. In some embodiments, a pharmaceutical formulation comprises any of the anti-Tau antibodies provided herein and a pharmaceutically acceptable carrier. In another embodiment, a pharmaceutical formulation comprises any of the anti-Tau antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent.

For example, the composition according to the invention may be administered in combination with other compositions comprising an additional therapeutic agent, such as a biologically active substance or compound such as, for example, a known compound used in the medication of tauopathies and/or of amyloidoses, a group of diseases and disorders associated with amyloid or amyloid-like protein such as the amyloid β protein involved in Alzheimer's Disease.

Generally, the other biologically active compound may include neuron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, serotonergic receptor antagonists, or other therapeutic agents. In particular, the biologically active agent or compound may comprise at least one compound selected from compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), secretase activators, beta- and gamma-secretase inhibitors, tau proteins, anti-Tau antibodies (including, but not limited to, antibodies disclosed in WO2012049570, WO2014028777, WO2014165271, WO2014100600, WO2015200806, U.S. Pat. Nos. 8,980,270, and 8,980,271), neurotransmitter, beta-sheet breakers, antiinflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs and nutritive supplements such as, for example, vitamin B 12, cysteine, a precursor of acetylcholine, lecithin, choline, Ginkgo biloba, acyetyl-L-carnitine, idebenone, propentofylline, or a xanthine derivative, together with a binding peptide according to the invention including antibodies, particularly monoclonal antibodies and active fragments thereof, and, optionally, a pharmaceutically acceptable carrier and/or a diluent and/or an excipient and instructions for the treatment of diseases.

In some embodiments, an antibody of the invention may be administered in combination with a neurological drug. Such neurological drugs include, but are not limited to, an antibody or other binding molecule (including, but not limited to a small molecule, a peptide, an aptamer, or other protein binder) that specifically binds to a target selected from: beta secretase, presenilin, amyloid precursor protein or portions thereof, amyloid beta peptide or oligomers or fibrils thereof, death receptor 6 (DR6), receptor for advanced glycation endproducts (RAGE), parkin, and huntingtin; an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an, an interferon, and a glucocorticoid or corticosteroid. The term “corticosteroid” includes, but is not limited to, fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, and triamcinolone acetonide.

In some embodiments, one or more anti-amyloid beta (anti-Abeta) antibodies may be administered with an anti-Tau antibody provided herein. Non-limiting examples of such anti-Abeta antibodies include crenezumab, solanezumab, bapineuzumab, aducanumab, gantenerumab, and BAN-2401 (Biogen, Eisai Co., Ltd.). In some embodiments, one or more beta-amyloid aggregation inhibitors may be administered with an anti-Tau antibody provided herein. Nonlimiting exemplary beta-amyloid aggregation inhibitors include ELND-005 (also referred to as AZD-103 or scyllo-inositol), tramiprosate, and PTI-80 (Exebiyl-1®; ProteoTech). In some embodiments, one or more BACE inhibitors may be administered with an anti-Tau antibody provided herein. Non-limiting examples of such BACE inhibitors include E-2609 (Biogen, Eisai Co., Ltd.), AZD3293 (also known as LY3314814; AstraZeneca, Eli Lilly & Co.), MK-8931 (verubecestat), and JNJ-54861911 (Janssen, Shionogi Pharma). In some embodiments, one or more Tau inhibitors may be administered with an anti-Tau antibody provided herein. Non-limiting examples of such Tau inhibitors include methylthioninium, LMTX (also known as leuco-methylthioninium or Trx-0237; TauRx Therapeutics Ltd.), Rember™ (methylene blue or methylthioninium chloride [MTC]; Trx-0014; TauRx Therapeutics Ltd), PBT2 (Prana Biotechnology), and PTI-51-CH3 (TauPro™; ProteoTech). In some embodiments, one or more other anti-Tau antibodies may be administered with an anti-Tau antibody provided herein. Non-limiting examples of such other anti-Tau antibodies include BIIB092 or BMS-986168 (Biogen, Bristol-Myers Squibb) and ABBV-8E12 or C2N-8E12 (AbbVie, C2N Diagnostics, LLC). In some embodiments, a general misfolding inhibitor, such as NPT088 (NeuroPhage Pharmaceuticals), may be administered with an anti-Tau antibody provided herein.

In some embodiments, the composition according to the invention may comprise niacin or memantine together with a chimeric antibody or a humanized antibody according to the invention including antibodies, particularly monoclonal antibodies and active fragments thereof, and, optionally, a pharmaceutically acceptable carrier and/or a diluent and/or an excipient.

In some embodiments, compositions are provided that comprise “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with the chimeric antibody or the humanized antibody according to the invention or active fragments thereof, and, optionally, a pharmaceutically acceptable carrier and/or a diluent and/or an excipient.

Other compounds that can be suitably used in compositions in addition to chimeric antibody or humanized antibody according to the invention, are those disclosed, for example, in WO 2004/058258 (see especially pages 16 and 17) including therapeutic drug targets (page 36-39), alkanesulfonic acids and alkanolsulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptors (PPAR) agonists (pages 63-67), cholesterol-lowering agents (pages 68-75); amyloid inhibitors (pages 75-77), amyloid formation inhibitors (pages 77-78), metal chelators (pages 78-79), anti-psychotics and anti-depressants (pages 80-82), nutritional supplements (pages 83-89) and compounds increasing the availability of biologically active substances in the brain (see pages 89-93) and prodrugs (pages 93 and 94), which document is incorporated herein by reference, but especially the compounds mentioned on the pages indicated above.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In some embodiments, administration of the anti-Tau antibody and administration of an additional therapeutic agent occur within about one month, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various timepoints, bolus administration, and pulse infusion are contemplated herein.

In some embodiments, a dose of MTAU or hMTAU is administered. In some embodiments, the dose of MTAU or hMTAU administered is between 225 mg and 16800 mg. In some embodiments, the dose of MTAU or hMTAU administered is 225 mg, 675 mg, 1200 mg, 1500 mg, 2100 mg, 4200 mg, 4500 mg, 8100 mg, 8400 mg, or 16800 mg.

In some embodiments, the dose of MTAU or hMTAU administered is 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, or 8500 mg.

In some embodiments, the dose of MTAU or hMTAU administered is 200 mg, 700 mg, 1200 mg, 1500 mg, 1700 mg, 2200 mg, 2700 mg, 3200 mg, 3700 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 15500 mg, 16000 mg, or 16500 mg.

In some embodiments, the dose of MTAU or hMTAU administered is 225 mg, 725 mg, 1225 mg, 1725 mg, 2225 mg, 2725 mg, 3225 mg, 3725 mg, 4225 mg, 4725 mg, 5225 mg, 5725 mg, 6225 mg, 6725 mg, 7225 mg, 7725 mg, 8225 mg, 8725 mg, 9225 mg, 9725 mg, 10225 mg, 10725 mg, 11225 mg, 11725 mg, 12225 mg, 12725 mg, 13225 mg, 13725 mg, 14225 mg, 14725 mg, 15225 mg, 15725 mg, 16225 mg, or 16725 mg.

In some embodiments, the dose of MTAU or hMTAU administered is between 1000 mg and 2000 mg. In some embodiments, the dose of MTAU or hMTAU administered is between 4000 mg and 16800 mg. In some embodiments, the dose of MTAU or hMTAU administered is between 4000 mg and 5000 mg. In some embodiments, the dose of MTAU or hMTAU administered is between 4000 mg and 4500 mg. In some embodiments, the dose of MTAU or hMTAU administered is between 4500 mg and 5000 mg. In some embodiments, the dose of MTAU or hMTAU administered is between 5000 mg and 5500 mg. In some embodiments, the dose of MTAU or hMTAU administered is between 5500 mg and 6000 mg. In some embodiments, the dose of MTAU or hMTAU administered is between 6000 mg and 6500 mg. In some embodiments, the dose of MTAU or hMTAU administered is between 6500 mg and 7000 mg. In some embodiments, the dose of MTAU or hMTAU administered is between 7000 mg and 7500 mg. In some embodiments, the dose of MTAU or hMTAU administered is between 7500 mg and 8000 mg. In some embodiments, the dose of MTAU or hMTAU administered is between 8000 mg and 8500 mg. In some embodiments, the dose of MTAU or hMTAU administered is between 8500 mg and 9000 mg. In some embodiments, the dose of MTAU or hMTAU administered is between 9000 mg and 9500 mg. In some embodiments, the dose of MTAU or hMTAU administered is between 9500 mg and 10000 mg. In some embodiments, the dose of MTAU or hMTAU administered is between 10000 mg and 10500 mg. In some embodiments, the dose of MTAU or hMTAU administered is between 10500 mg and 11000 mg. In some embodiments, the dose of MTAU or hMTAU administered is between 11000 mg and 11500 mg. In some embodiments, the dose of MTAU or hMTAU administered is between 11500 mg and 12000 mg. In some embodiments, the dose of MTAU or hMTAU administered is between 12000 mg and 12500 mg. In some embodiments, the dose of MTAU or hMTAU administered is between 12500 mg and 13000 mg. In some embodiments, the dose of MTAU or hMTAU administered is between 13000 mg and 13500 mg. In some embodiments, the dose of MTAU or hMTAU administered is between 13500 mg and 14000 mg. In some embodiments, the dose of MTAU or hMTAU administered is between 14000 mg and 14500 mg. In some embodiments, the dose of MTAU or hMTAU administered is between 14500 mg and 15000 mg. In some embodiments, the dose of MTAU or hMTAU administered is between 15000 mg and 15500 mg. In some embodiments, the dose of MTAU or hMTAU administered is between 15500 mg and 16000 mg. In some embodiments, the dose of MTAU or hMTAU administered is between 16000 mg and 16500 mg. In some embodiments, the dose of MTAU or hMTAU administered is between 16500 mg and 17000 mg.

In some embodiments, the dose of hMTAU administered is between 50 mg/kg and 240 mg/kg. In some embodiments, the dose of hMTAU administered is between 60 mg/kg and 120 mg/kg. In some embodiments, the dose of hMTAU administered is between 60 mg/kg and 70 mg/kg. In some embodiments, the dose of hMTAU administered is between 70 mg/kg and 80 mg/kg. In some embodiments, the dose of hMTAU administered is between 80 mg/kg and 90 mg/kg. In some embodiments, the dose of hMTAU administered is between 90 mg/kg and 100 mg/kg. In some embodiments, the dose of hMTAU administered is between 100 mg/kg and 110 mg/kg. In some embodiments, the dose of hMTAU administered is between 110 mg/kg and 120 mg/kg. In some embodiments, the dose of hMTAU administered is between 2.5 mg/kg and 5 mg/kg, 5 mg/kg and 10 mg/kg, 10 mg/kg and 15 mg/kg, 15 mg/kg and 20 mg/kg, 20 mg/kg and 30 mg/kg, 30 mg/kg and 40 mg/kg, 40 mg/kg and 50 mg/kg, or 50 mg/kg and 60 mg/kg.

In some embodiments, the dose of hMTAU administered is 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, or 180 mg/kg. In some embodiments, the dose of hMTAU administered is 150 mg/kg. In some embodiments, the dose of hMTAU administered is 180 mg/kg.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. As used herein, a “split dose” is the division of single unit dose or total daily dose into two or more doses, e.g., two or more administrations of the single unit dose. The antibody may be administered as “split dose.”

The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate. In some embodiments, the antibody is provided in a formulation for immediate release and the other agent is formulated for extended release or vice versa.

The antibody is suitably administered to the patient at one time or over a series of treatments. In some embodiments, the hMTAU is administered once every 4 weeks. In some embodiments, the hMTAU is administered once every 1, 2, 4, 5, 6, 7, or 8 weeks. In some embodiments, the hMTAU is administered 2, 3, 4, 5, 6, 7, 8, 9, 10, 11, 12, 13, or 14 times every 1, 2, 4, 5, 6, 7, or 8 weeks.

In some embodiments, the hMTAU is administered intravenously. In some embodiments, the hMTAU is administered subcutaneously. In some embodiments, the hMTAU is administered subcutaneously at a dose of 1200 mg. In some embodiments, the hMTAU is administered intravenously at a dose of 225 mg, 675 mg, 1500 mg, 2100 mg, 4200 mg, 4500 mg, 8100 mg, 8400 mg, or 16800 mg.

G. Monitoring/Assessing Response to Therapeutic Treatment

In some embodiments, a patient being treated with an antibody described herein is monitored or assessed to determine if the patient is benefiting from the treatment. In some embodiments, a therapeutic benefit is a slowing, delay, or cessation of the progression of AD, or a reduction in clinical, functional, or cognitive decline. For example, benefits may include, but are not limited to, (1) inhibition, to some extent, of disease progression, including slowing of progression and complete arrest; (2) reduction in amount of plaque or reduction in brain amyloid accumulation and/or reduction in neurofibrillary tangles and/or reduction in brain tau accumulation; (3) improvement in one or more assessment metrics, including but not limited to CDR-SB, RBANS, and/or ADAS-Cog 13 scores; (4) improvement in daily functioning of the patient; (5) a decrease in a biomarker indicative of AD, such as Abeta or tau in cerebrospinal fluid; and (6) an increase in a biomarker indicative of improvement of AD. Patients may be assessed using any measure that can detect a benefit to the patient.

In some embodiments, the cognitive ability and/or daily functioning of the patient is assessed prior to, during, and/or after a course of therapy with an antibody described herein. Various cognitive and functional assessment tools are known in the art, and may be used to assess, diagnose, and/or score mental function, cognition, and/or neurological deficit. Exemplary tools include, but are not limited to, ADAS-Cog (including ADAS-Cog 12, ADAS-Cog 13, and ADAS-Cog 14), CDR-SB, MMSE, Instrumental Activities of Daily Living (iADL), Alzheimer's Disease Cooperative Study Group-Activities of Daily Living Inventory (ADCS-ADL), and RBANS.

In some embodiments, a patient that has been treated with an antibody described herein shows an improvement (i.e., a decrease) in the patient's CDR-SB score compared to baseline (e.g., prior to treatment or at an earlier timepoint during treatment). In some embodiments, the patient's CDR-SB score decreases by at least 15%, or by at least 20%, or by at least 25%, or by at least 30%, or by at least 35%, or by at least 40%, or by at least 45%, or by at least 50%. In some embodiments, a patient that has been treated with an antibody described herein shows a slowing of the rate of increase in their CDR-SB score by at least 15%, or by at least 20%, or by at least 25%, or by at least 30%, or by at least 35%, or by at least 40%, or by at least 45%, or by at least 50%. In some embodiments, a stable CDR-SB score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In some embodiments, a patient that has been treated with an antibody described herein shows an improvement (i.e., an increase) in the patient's RBANS score compared to baseline (e.g., prior to treatment or at an earlier timepoint during treatment). In some embodiments, the patient's RBANS score increases by at least 15%, or by at 20%, or by at least 25%, or by at least 30%, or by at least 35%, or by at least 40%, or by at least 45%, or by at least 50%. In some embodiments, a patient that has been treated with an antibody described herein shows a slowing of the rate of decrease in their RBANS score by at least 15%, at least 20%, or by at least 25%, or by at least 30%, or by at least 35%, or by at least 40%, or by at least 45%, or by at least 50%. In some embodiments, a stable RBANS score compared to baseline (e.g., prior to treatment or at an earlier timepoint during treatment) may be indicative of a slowing, delay, or cessation of the progression of AD, or a reduction in clinical or cognitive decline.

In some embodiments, a patient that has been treated with an antibody described herein shows an improvement (i.e., a decrease) in the patient's ADAS-Cog score (such as ADAS-Cog 13) compared to baseline (e.g., prior to treatment or at an earlier timepoint during treatment). In some embodiments, the patient's ADAS-Cog score decreases by at least 15%, or by at least 20%, or by at least 25%, or by at least 30%, or by at least 35%, or by at least 40%, or by at least 45%, or by at least 50%. In some embodiments, a patient that has been treated with an antibody described herein shows a slowing of the rate of increase in their ADAS-Cog 13 score by at least 15%, or by at least 20%, or by at least 25%, or by at least 30%, or by at least 35%, or by at least 40%, or by at least 45%, or by at least 50%. In some embodiments, a stable ADAS-Cog 13 score compared to baseline (e.g., prior to treatment or at an earlier timepoint during treatment) may be indicative of a slowing, delay, or cessation of the progression of AD, or a reduction in clinical or cognitive decline.

In various embodiments, the patient is assessed at least 13 weeks, at least 24 weeks, at least 25 weeks, at least 37 weeks, at least 49 weeks, at least 61 weeks, at least 69 weeks, at least 73 weeks, at least 85 weeks, at least 97 weeks, at least 109 weeks, at least 121 weeks, at least 133 weeks, at least 145 weeks, at least 157 weeks, or at least 169 weeks after the beginning treatment with an antibody described herein. In various embodiments, the baseline score is prior to a treatment provided herein. “Prior to treatment” as used herein, means at any time from diagnosis of the disease (such as AD) up to administration of the treatment provided herein. In some embodiments, prior to treatment is within 12 months, 6 months, 3 months, 2 months, 1 month, 3 weeks, 2 weeks, or 1 week prior to treatment. In some embodiments, baseline is at an earlier timepoint during a treatment provided herein. In some embodiments, baseline and the time point at which the patient is assessed for therapeutic benefit are at least 13 weeks, at least 24 weeks, at least 25 weeks, at least 37 weeks, at least 49 weeks, at least 61 weeks, at least 69 weeks, at least 73 weeks, at least 85 weeks, at least 97 weeks, at least 109 weeks, at least 121 weeks, at least 133 weeks, at least 145 weeks, at least 157 weeks, or at least 169 weeks apart.

H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-Tau antibody.

### III. Examples

The following are examples of methods and compositions of the invention. It is understood that various other embodiments may be practiced, given the general description provided above.

### Example 1: Clinical Study to Evaluate the Safety and Tolerability of a Humanized Anti-Tau Monoclonal Antibody in Healthy Volunteers and Patients with Mild to Moderate Alzheimer's Disease

A Phase I randomized, placebo-controlled, double blind study was designed to evaluate the safety, tolerability, pharmacokinetics, and pharmacodynamics of a humanized anti-Tau monoclonal antibody (hMTAU) in healthy volunteers and patients diagnosed with mild to moderate Alzheimer's Disease (AD). Healthy volunteers were 18-80 years of age and without symptomatic cognitive decline. AD patients were 50-80 years of age, and had a diagnosis of probable AD (by NIA-AA criteria), with a Mini-Mental Status Examination (MMSE; Folstein et al., 1975, J Psychiatr. Res. 12:189-98) score of 16-28, a Clinical Dementia Rating (CDR; Morris, 1993, Neurology 43:2412-4) global score of 0.5, 1, or 2, and a ¹⁸F-florbetapir PET scan that was positive for cerebral amyloid according to a visual read.

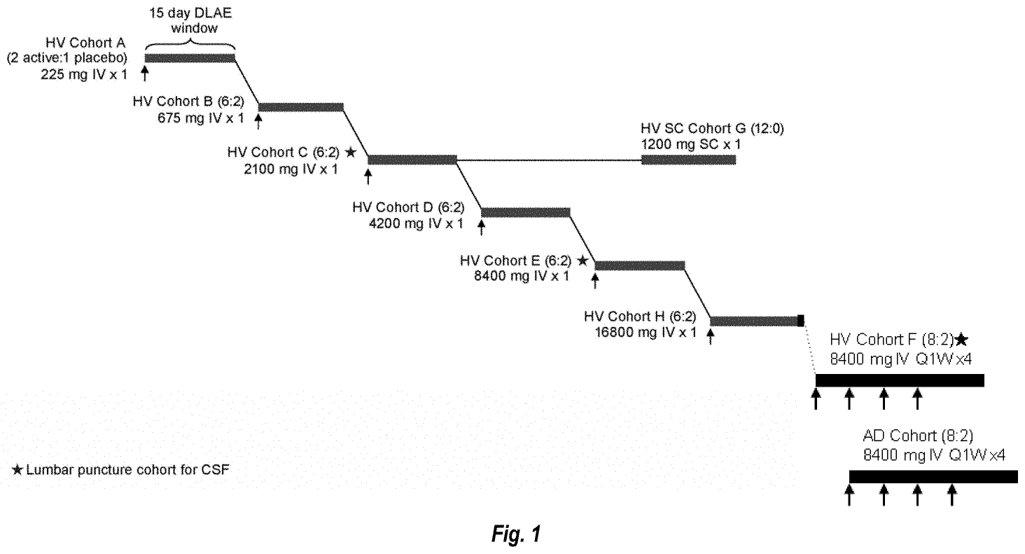

The study included a single-dose escalation (SD) stage in healthy volunteers followed by a multiple-dose stage (MD) in healthy volunteers and AD patients. The study included approximately seven SD cohorts in healthy volunteers administered hMTAU, either intravenously (IV) or subcutaneously (SC), one or more MD cohorts in healthy volunteers administered hMTAU intravenously every week (Q1W)×4, and one or more MD cohorts in participants with AD administered hMTAU intravenously Q1W×4. The single ascending dose study of healthy volunteers received single doses of hMTAU either intravenously (IV; doses ranging from 225 mg to 16,800 mg) or subcutaneously (SC; 1,200 mg). Each SD IV cohort included 6 healthy volunteers treated with active hMTAU and 2 participants treated with matching placebo, at a single-dose level. The SD SC cohort was designed to assess bioavailability and SC tolerability. The SD SC cohort included 12 healthy volunteer participants treated with active hMTAU; with no placebo group. The design of the SD stage is depicted in FIG. 1.

Approximately 10 healthy volunteers were enrolled in MD cohorts (IV administration), and approximately 10 participants with AD were enrolled in MD cohorts (IV administration). MD cohorts consisted of either healthy volunteers or participants with AD. Each MD IV cohort included 8 participants treated with active hMTAU and 2 participants treated with matching placebo. The MD cohorts in healthy volunteers and participants with AD were enrolled in a staggered, parallel fashion. Table 2 provides details of treatment cohorts.

TABLE 2

| | | | | | | | | | AD |
|---|---|---|---|---|---|---|---|---|---|
| | | | | | | | | | Cohort |
| | Cohort | Cohort | Cohort | Cohort | Cohort | | Cohort | Cohort F | 8400 |
| | A | B 675 | C 2100 | D 4200 | E 8400 | Cohort H | G 1200 | 8400 mg | mg |
| | 225 mg | mg IV | mg IV | mg IV | mg IV | 16800 mg | mg SC | IV QW | IV QW |
| | IV x1 | x1 | x1 | x1 | x1 | IV x1 | x1 | x4 | x4 |
| N | 3 (2:1) | 8 (6:2) | 8 (6:2) | 8 (6:2) | 8 (6:2) | 8 (6:2) | 12 (12:0) | 10 (8:2) | 10 (8:2) |
| (active:placebo) | | | | | | | | | |

Assessments were performed as follows. Clinical and safety assessments at screening and at prespecified timepoints during the study included: vital signs, physical and neurological examinations, laboratory testing, ECGs, serum and (in some cohorts) cerebrospinal fluid (CSF) pharmacokinetics, plasma and (in some cohorts) CSF tau levels. A Safety Monitoring Committee reviewed safety data on an ongoing basis, and made dose escalation decisions according to stopping rules and dose limiting adverse events, as defined in the protocol. For an event to be deemed a dose limiting adverse event (DLAE), it must have occurred during the DLAE window, have no other clearly attributable cause other than study drug, and be at least one of the following: serious; Grade 3 or higher; Grade 2 in a neurological category; or Grade 2 and infusion/injection-related.

Fifty-five (55) healthy volunteers were enrolled in the single dose cohorts. In the single ascending dose stage, preliminary data from early assessments showed that the most frequently reported adverse events (AEs) regardless of causality were headache (n=6), nausea (n=3), infusion site bruising (n=2) and hematoma (n=1), and urinary tract infection (n=3). AEs attributed to study treatment were injection site reactions (injection site bruising (n=2) and injection site pain (n=1). There were additional related AEs reported in one patient (ALT/AST increased; nausea; feeling abnormal; fatigue; headache; diarrhea; arthralgia). In assessment of single-dose cohorts with further timepoints, the data showed that adverse events were reported in >1 participant included headache (n=6), injection/infusion site reaction (n=6), upper respiratory tract infection (n=3), nausea (n=3), vomiting (n=2), and GI viral infection (n=2). In multi-dose cohorts, adverse events reported in >1 participant included vessel puncture site complications (n=2) and postural dizziness (n=2). Nine (12%) participants (all in single-dose cohorts) experienced an adverse event reported as related to study drug: Grade 1 injection site reaction was the only related adverse event in >1 participant (n=3, including 2 subjects reporting bruising and 1 reporting injection site pain; all three resolved without treatment). At the time of data assessment, thirty-two (43%) participants experienced an adverse event regardless of investigator-assessed causality: 23/55 (42%) in single-dose cohorts; 9/20 (45%) in multi-dose cohorts (40% of HVs and 50% of AD patients).

Overall, the study showed that hMTAU was well-tolerated. In a preliminary assessment during the study, AEs were non-serious Grade 1-2; there were no Grade ≥3 AEs reported. There were no dose-limiting adverse events (DLAEs) or AEs leading to withdrawal from treatment, or dose modifications or interruptions. One subject in Cohort D (4200 mg IV) withdrew from the study at Study Day 57 due to personal decision. There were no reported serious AEs or deaths on study, even at the highest dose tested. In conclusion, preliminary data showed that single IV and SC doses of hMTAU up to 16,800 mg were safe and well-tolerated in healthy volunteers.

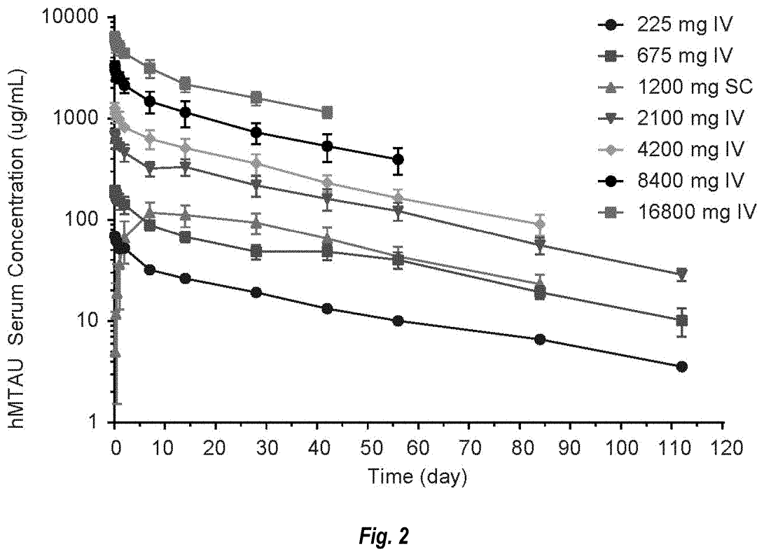

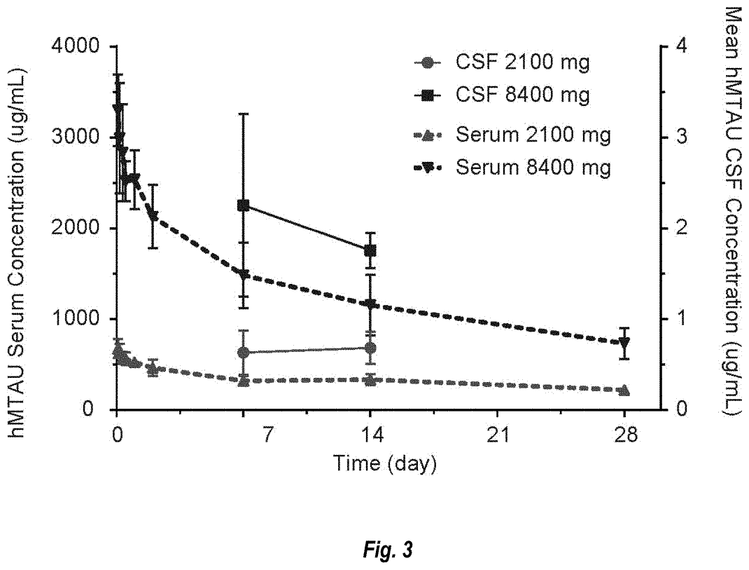

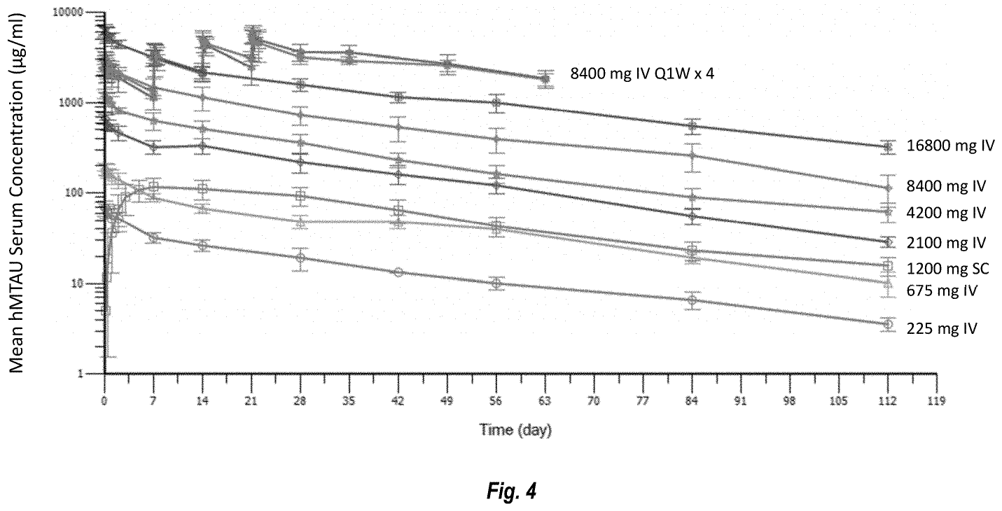

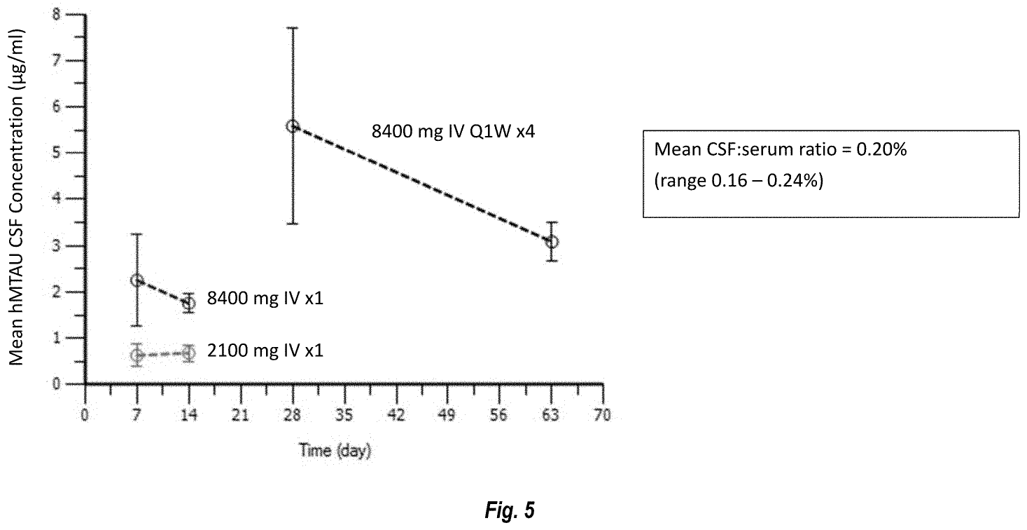

hMTAU exhibited a dose-proportional and 2-compartmental pharmacokinetics in serum across the dose range studied (225 mg-16,800 mg). The terminal half-life of hMTAU was ˜30 days: median hMTAU terminal t_{1/2} was 32.3 days (range 23-46 days; following single IV dose). FIG. 4. The bioavailability estimate for subcutaneous formulation (1200 mg) was ˜70%. hMTAU was detectable in CSF and had a CSF/serum % of 0.15-0.2%. FIGS. 2-5 show the mean concentration of hMTAU in serum (FIG. 2 and FIG. 4) and CSF (FIG. 3 and FIG. 5) after single dose administration at the doses and via the routes indicated. Overall, the pharmacokinetic properties of hMTAU exhibited dose-proportionality, with no evidence of target-mediated drug disposition. In addition, hMTAU was detectable in CSF, establishing CNS exposure to the antibody.

Thus, hMTAU was safe and well-tolerated at single doses up to 16,800 mg IV in human volunteers and at multiple doses of 8,400 mg Q1W×4 in human volunteers and patients with AD. hMTAU exhibited dose-proportional PK and a median t_{1/2} of 32.3 days and SC bioavailability of approximately 70%. hMTAU was detectable in CSF, indicating CNS exposure.

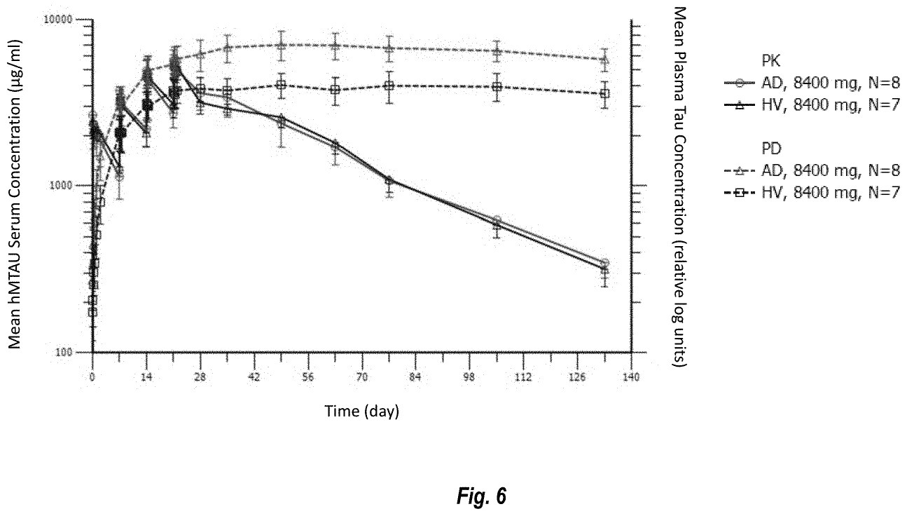

Pharmacokinetic (PK) and pharmacodynamic (PD) profiles were assessed in eight (8) Alzheimer's disease (AD) patients and seven (7) healthy volunteers (HV) after four weekly doses of hMTAU at 8,400 mg IV. One healthy volunteer was administered an incorrect dose of 4200 mg hMTAU and was removed from the exposure analysis. As shown in FIG. 6, preliminary results indicated that, despite similar exposure to hMTAU, a more robust PD response was seen in Alzheimer's disease (AD) patients compared to healthy volunteers (HV). Compared to HVs, AD patients exhibited two-fold greater levels of plasma tau following hMTAU administration. The PK response was similar between AD patients and HVs. Preliminary studies showed that baseline plasma Tau levels were higher in AD patients (Avg.=26.3536 pg/mL; median=24.806 pg/mL) as compared to HVs (Avg.=16.4181 pg/mL; median=15.0045 pg/mL).

### Example 2: Clinical Study to Evaluate Three Different Doses of a Humanized Anti-Tau Monoclonal Antibody in Patients with Prodromal to Mild Alzheimer's Disease

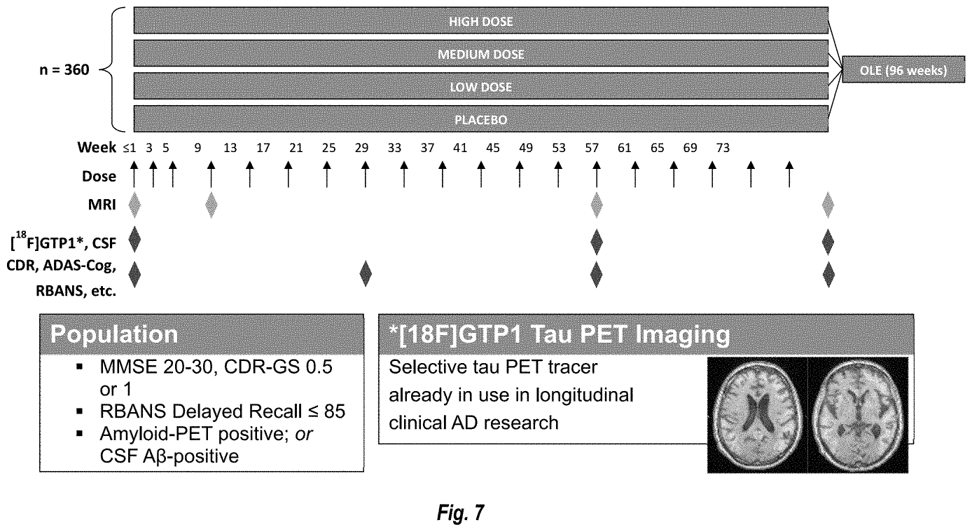

FIG. 7 shows a study design for a clinical study to evaluate three different doses of a humanized anti-Tau monoclonal antibody (hMTAU) in prodromal to mild Alzheimer's disease patients. 360 patients are enrolled, with the following enrollment criteria: one or more of (1) a Mini Mental State Examination (MMSE) score of 20-30; (2) a Clinical Dementia Rating-Global Score (CDR-GS) of 0.5 or 1; (3) a Repeatable Battery for the Assessment of Neuropsychological Status (RBANS) delayed recall score of ≤85; and/or (4) amyloid-PET positive or CSF AP positive. hMTAU is administered in weeks 1, 3, and 5, and then approximately every 4 weeks thereafter. During the course of the study, patients undergo one or more of MRI, [¹⁸F]GTP1 Tau PET imaging, MMSE, CDR-GS, RBANS, and CSF screening.

Although the foregoing invention has been described in some detail by way of illustration and example for purposes of clarity of understanding, the descriptions and examples should not be construed as limiting the scope of the invention. Unless expressly indicated otherwise, all ranges described herein (e.g., “between 4000 mg and 16800 mg”) encompass the endpoints of the range. The disclosures of all patent and scientific literature cited herein are expressly incorporated in their entirety by reference.

TABLE OF SEQUENCES

| SEQ | | |
|---|---|---|
| ID | | |
| NO | Description | Sequence |
| 2 | Human Tau epitope (2- | AEPRQEFEVMEDHAGTYGLGDRK |
| | 24) | |
| 4 | Cynomolgus monkey Tau | AEPRQEFDVMEDHAGTYGLGDRK |
| | epitope (2-24) | |
| 10 | MTAU heavy chain | EVQLVESGGD LAKPGGSLKL SCTASGLIFR SYGMSWVRQT |
| | variable region (VH) | PDKRLEWVAT INSGGTYTYY PDSVKGRFTI SRDNAKNTLY |
| | | LQMSSLKSED TAMYYCANSY SGAMDYWGQG TSVTVSS |
| 11 | MTAU light chain | DDLLTQTPLS LPVSLGDPAS ISCRSSQSIV HSNGNTYFEW |
| | variable region (VL) | YLQKPGQSPK LLIYKVSNRF SGVPDRFSGS GSGTDFTLKI |
| | | SRVEAEDLGV YYCFQGSLVP WTFGGGTKLE IK |
| 12 | MTAU HVR-H1 | SYGMS |
| 13 | MTAU HVR-H2 | TINSGGTYTYYPDSVKG |
| 14 | MTAU HVR-H3 | SYSGAMDY |
| 15 | MTAU HVR-L1 | RSSQSIVHSNGNTYFE |
| 16 | MTAU HVR-L2 | KVSNRFS |
| 17 | MTAU HVR-L3 | FQGSLVPWT |
| 18 | hMTAU heavy chain | EVQLVESGGG LVQPGGSLRL SCAASGLIFR SYGMSWVRQA |
| | variable region (VH) | PGKGLEWVAT INSGGTYTYY PDSVKGRFTI SRDNSKNTLY |
| | | LQMNSLRAED TAVYYCANSY SGAMDYWGQG TLVTVSS |
| 19 | hMTAU light chain | DDVLTQTPLS LPVTPGQPAS ISCRSSQSIV HSNGNTYLEW |
| | variable region (VL) | YLQKPGQSPQ LLIYKVSNRF SGVPDRFSGS GSGTDFTLKI |
| | | SRVEAEDVGV YYCFQGSLVP WTFGQGTKVE IK |
| 20 | hMTAU HVR-H1 | SYGMS |
| 21 | hMTAU HVR-H2 | TINSGGTYTYYPDSVKG |
| 22 | hMTAU HVR-H3 | SYSGAMDY |
| 23 | hMTAU HVR-L1 | RSSQSIVHSNGNTYLE |
| 24 | hMTAU HVR-L2 | KVSNRFS |
| 25 | hMTAU HVR-L3 | FQGSLVPWT |
| 26 | hMTAU heavy chain | EVQLVESGGG LVQPGGSLRL SCAASGLIFR SYGMSWVRQA |
| | version 1 | PGKGLEWVAT INSGGTYTYY PDSVKGRFTI SRDNSKNTLY |
| | | LQMNSLRAED TAVYYCANSY SGAMDYWGQG TLVTVSSAST |
| | | KGPSVFPLAP CSRSTSESTA ALGCLVKDYF PEPVTVSWNS |
| | | GALTSGVHTF PAVLQSSGLY SLSSVVTVPS SSLGTKTYTC |
| | | NVDHKPSNTK VDKRVESKYG PPCPPCPAPE FLGGPSVFLF |
| | | PPKPKDTLYI TREPEVTCVV VDVSQEDPEV QFNWYVDGVE |
| | | VHNAKTKPRE EQFNSTYRVV SVLTVLHQDW LNGKEYKCKV |
| | | SNKGLPSSIE KTISKAKGQP REPQVYTLPP SQEEMTKNQV |
| | | SLTCLVKGFY PSDIAVEWES NGQPENNYKT TPPVLDSDGS |
| | | FFLYSRLTVD KSRWQEGNVF SCSVMHEALH NHYTQKSLSL SLGK |
| 27 | hMTAU heavy chain | EVQLVESGGG LVQPGGSLRL SCAASGLIFR SYGMSWVRQA |
| | version 2 | PGKGLEWVAT INSGGTYTYY PDSVKGRFTI SRDNSKNTLY |
| | | LQMNSLRAED TAVYYCANSY SGAMDYWGQG TLVTVSSAST |
| | | KGPSVFPLAP CSRSTSESTA ALGCLVKDYF PEPVTVSWNS |
| | | GALTSGVHTF PAVLQSSGLY SLSSVVTVPS SSLGTKTYTC |
| | | NVDHKPSNTK VDKRVESKYG PPCPPCPAPE FLGGPSVFLF |
| | | PPKPKDTLYI TREPEVTCVV VDVSQEDPEV QFNWYVDGVE |
| | | VHNAKTKPRE EQFNSTYRVV SVLTVLHQDW LNGKEYKCKV |
| | | SNKGLPSSIE KTISKAKGQP REPQVYTLPP SQEEMTKNQV |
| | | SLTCLVKGFY PSDIAVEWES NGQPENNYKT TPPVLDSDGS |
| | | FFLYSRLTVD KSRWQEGNVF SCSVMHEALH NHYTQKSLSL SLG |
| 28 | hMTAU light chain | DDVLTQTPLS LPVTPGQPAS ISCRSSQSIV HSNGNTYLEW |
| | | YLQKPGQSPQ LLIYKVSNRF SGVPDRFSGS GSGTDFTLKI |
| | | SRVEAEDVGV YYCFQGSLVP WTFGQGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |

1. A method of treating a tauopathy comprising administering to an individual with a tauopathy a monoclonal antibody that binds human tau at a dose between 225 mg and 16800 mg, wherein the antibody comprises HVR-H1 comprising the amino acid sequence of SEQ ID NO: 20; HVR-H2 comprising the amino acid sequence of SEQ ID NO: 21; HVR-H3 comprising the amino acid sequence of SEQ ID NO: 22; HVR-L1 comprising the amino acid sequence of SEQ ID NO: 23; HVR-L2 comprising the amino acid sequence of SEQ ID NO: 24; and HVR-L3 comprising the amino acid sequence of SEQ ID NO: 25.

2. The method of claim 1, wherein the method comprises administering the antibody at a dose of 225 mg, 675 mg, 1200 mg, 1500 mg, 2100 mg, 4200 mg, 4500 mg, 8100 mg, 8400 mg, or 16800 mg.

3. The method of claim 1, wherein the method comprises administering the antibody at a dose between 4000 mg and 16800 mg.

4. The method of claim 1, wherein the method comprises administering the antibody at a dose between 4000 mg and 8500 mg.

5. The method of claim 1, wherein the method comprises administering the antibody at a dose between 225 mg and 600 mg, 600 mg and 1000 mg, 1000 mg and 2000 mg, 2000 mg and 3000 mg, 3000 mg and 4000 mg, 4000 mg and 4500 mg, 4000 mg and 5000 mg, 4500 mg and 5000 mg, 5000 mg and 5500 mg, 5500 mg and 6000 mg, 6000 mg and 6500 mg, 6500 mg and 7000 mg, 7000 mg and 7500 mg, 7500 mg and 8000 mg, or 8000 mg and 8500 mg.

6. The method of claim 1, wherein the method comprises administering the antibody at a dose between 50 mg/kg and 240 mg/kg.

7. The method of claim 1, wherein the method comprises administering the antibody at a dose between 60 mg/kg and 120 mg/kg.

8. The method of claim 1, wherein the method comprises administering the antibody at a dose between 2.5 mg/kg and 5 mg/kg, 5 mg/kg and 10 mg/kg, 10 mg/kg and 15 mg/kg, 15 mg/kg and 20 mg/kg, 20 mg/kg and 30 mg/kg, 30 mg/kg and 40 mg/kg, 40 mg/kg and 50 mg/kg, 50 mg/kg and 60 mg/kg, 60 mg/kg and 70 mg/kg, 70 mg/kg and 80 mg/kg, 80 mg/kg and 90 mg/kg, 90 mg/kg and 100 mg/kg, 100 mg/kg and 110 mg/kg, or 110 mg/kg and 120 mg/kg.

9. The method of claim 1, wherein the method comprises administering the antibody once or twice every 1, 2, 4, 5, 6, 7, or 8 weeks.

10. (canceled)

11. The method of claim 1, wherein the method comprises administering the antibody subcutaneously or intravenously.

12. (canceled)

13. (canceled)

14. The method of claim 1, wherein the tauopathy is a neurodegenerative tauopathy.

15. The method of claim 1, wherein the tauopathy is selected from Alzheimer's Disease, amyotrophic lateral sclerosis, Parkinson's disease, Creutzfeldt-Jacob disease, Dementia pugilistica, Down's Syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallervorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy.

16. (canceled)

17. The method of claim 15, wherein the tauopathy is Alzheimer's Disease (AD).

18. The method of claim 17, wherein the AD is early, prodromal, prodromal to mild, mild, mild to moderate or moderate.

19. The method of claim 1, wherein the treating comprises slowing memory loss or retaining or increasing memory capacity, memory function, or cognitive function in the individual.

20. The method of claim 19, wherein memory capacity, memory function, cognitive function, or memory loss is assessed using one or more of Clinical Dementia Rating-Sum of Boxes (CDR-SB), Repeatable Battery for the Assessment of Neuropsychological Status (RBANS), and Alzheimer's Disease Assessment Scale-Cognitive Subscale (ADAS-Cog).

21. The method of claim 20, wherein ADAS-Cog is ADAS-Cog 13.

22. The method of claim 20, wherein a decrease in CDR-SB score, or an increase in RBANS score, or a decrease in ADS-Cog score following administration of one or more doses of the antibody indicates one or more of increased memory capacity, memory function, or cognitive function in the individual.

23. The method of claim 20, wherein a stable CDR-SB score, RBANS score, or ADAS-Cog score, a slowing of the rate of increase of a CDR-SB score or ADAS-Cog score, or a slowing of the rate of decrease of a RBANS score following administration of one or more doses of the antibody indicates one or more of slowed memory loss or retained memory capacity, memory function, or cognitive function in the individual.

24. The method of claim 20, wherein the CDR-SB score, RBANS score, or ADAS-Cog score is compared to the respective score at baseline.

25. (canceled)

26. The method of claim 20, wherein the memory capacity, memory function, cognitive function, or memory loss is assessed at least 13 weeks, at least 24 weeks, at least 25 weeks, at least 37 weeks, at least 49 weeks, at least 61 weeks, at least 69 weeks, at least 73 weeks, at least 85 weeks, at least 97 weeks, at least 109 weeks, at least 121 weeks, at least 133 weeks, at least 145 weeks, at least 157 weeks, or at least 169 weeks after the beginning of treatment with the antibody.

27. A method of retaining or increasing one or more of memory capacity, memory function, or cognitive function or slowing memory loss in an individual, comprising administering to an individual with a tauopathy a monoclonal antibody that binds human tau at a dose between 225 mg and 16800 mg, wherein the antibody comprises HVR-H1 comprising the amino acid sequence of SEQ ID NO: 20; HVR-H2 comprising the amino acid sequence of SEQ ID NO: 21; HVR-H3 comprising the amino acid sequence of SEQ ID NO: 22; HVR-L1 comprising the amino acid sequence of SEQ ID NO: 23; HVR-L2 comprising the amino acid sequence of SEQ ID NO: 24; and HVR-L3 comprising the amino acid sequence of SEQ ID NO: 25.

28.-34. (canceled)

35. A method of reducing the level of Tau protein, non-phosphorylated Tau protein, phosphorylated Tau protein, or hyperphosphorylated Tau protein in an individual, comprising administering to an individual with a tauopathy a monoclonal antibody that binds human tau at a dose between 225 mg and 16800 mg, wherein the antibody comprises HVR-H1 comprising the amino acid sequence of SEQ ID NO: 20; HVR-H2 comprising the amino acid sequence of SEQ ID NO: 21; HVR-H3 comprising the amino acid sequence of SEQ ID NO: 22; HVR-L1 comprising the amino acid sequence of SEQ ID NO: 23; HVR-L2 comprising the amino acid sequence of SEQ ID NO: 24; and HVR-L3 comprising the amino acid sequence of SEQ ID NO: 25.

36.-53. (canceled)

54. The method of claim 1, wherein the method comprises administering at least one additional therapy.

55. The method of claim 54, wherein the additional therapy is selected from neurological drugs, corticosteroids, antibiotics, antiviral agents, anti-Tau antibodies, Tau inhibitors, anti-amyloid beta antibodies, beta-amyloid aggregation inhibitors, anti-BACE1 antibodies, and BACE1 inhibitors.

56. The method of claim 1, wherein the antibody comprises a heavy chain variable region comprising the amino acid sequence of SEQ ID NO: 18 and a light chain variable region comprising the amino acid sequence of SEQ ID NO: 19.

57. The method of claim 1, wherein the antibody comprises a heavy chain comprising the amino acid sequence of SEQ ID NO: 26 or SEQ ID NO: 27 and a light chain comprising the amino acid sequence of SEQ ID NO: 28.

58. The method of claim 1, wherein the antibody comprises a heavy chain consisting of the amino acid sequence of SEQ ID NO: 26 or SEQ ID NO: 27 and a light chain consisting of the amino acid sequence of SEQ ID NO: 28.

59. The method of claim 56, wherein the antibody is an IgG1 or an IgG4 antibody.

60. The method of claim 59, wherein the antibody is an IgG4 antibody.

61. The method of claim 60, wherein the antibody comprises M252Y, S254T, and T256E mutations.

62. The method of claim 61, wherein the antibody comprises an S228P mutation.

63. The method of claim 1, wherein the antibody is an antibody fragment.

64. The method of claim 1, wherein the antibody binds each of monomeric Tau, phosphorylated Tau, non-phosphorylated Tau, and oligomeric Tau with a K_{D} of less than 100 nM, less than 75 nM, or less than 50 nM.

65. The method of claim 1, wherein the antibody binds cynomolgus monkey Tau (SEQ ID NO: 4).

66. (canceled)

67. (canceled)
